(19) United States
(12) Patent Application Publication
(10) Pub. No.: US 2025/0084089 A1
(43) Pub. Date: Mar. 13, 2025

(54) Unique and Selective Targeting of CDK Activity in Aggressive Carcinomas

(71) Applicant: University of Windsor, Windsor (CA)

(72) Inventors: John F. Trant, LaSalle, CA (US); Lisa A. Porter, Amherstburg (CA); Bre-Anne Fifield, Windsor (CA); Daniel Meister, Windsor (CA); John J. Hayward, Windsor (CA); Lavleen Mader, LaSalle (CA); Maria Teodora Secara, Windsor (CA)

(21) Appl. No.: 18/821,262

(22) Filed: Aug. 30, 2024

Related U.S. Application Data
(60) Provisional application No. 63/580,052, filed on Sep. 1, 2023.

(51) Int. Cl. C07D 487/04; A61K 31/519; G01N 33/574

(52) U.S. Cl. CPC C07D 487/04; A61K 31/519; G01N 33/57484; G01N 2333/912

(57) ABSTRACT

In a preferred embodiment, there is provided a compound having structural Formula I, II, III or IV.

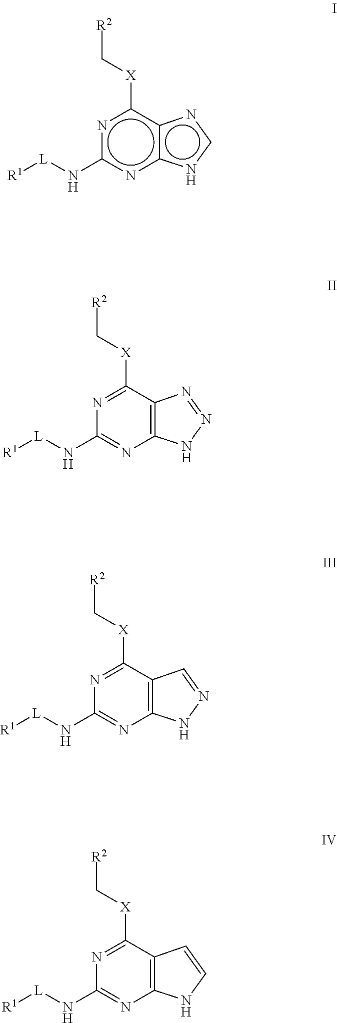

### SCOPE OF THE INVENTION

The present invention relates to a compound for treating cancer, and which includes a hydrophobic moiety preferably selected for binding to an active site of a cyclin-dependent kinase, Cdk1 or Cdk2 activated by Spy1.

### BACKGROUND OF THE INVENTION

There are many advanced, aggressive forms of cancer that currently lack effective therapeutic options. Combination chemotherapy, targeting different mechanisms such as slowing down cell proliferation or inducing damage to the DNA, is the current standard of care. As tumours progress, they accumulate mutations making them increasingly aggressive and resistant to therapies. Many of these mutations cripple the normal protective cellular mechanisms that halt cell growth and trigger the death of cells with damaged DNA. Reinstalling these protective pathways represents an attractive mechanism to sensitize some of the most aggressive cancer cells to treatment.

One family of protective proteins lost or blocked by aggressive cancers are Cyclin Dependent Kinase Inhibitors (CKIs). Basic research and pharma development have led to synthetic CKIs, which have met with variable success in the clinic.

### SUMMARY OF THE INVENTION

It is a non-limiting object of the present invention to provide a compound for treating cancer, and which may permit improved selectivity for a Spy1-Cdk complex or an active site thereof over, for example, a cyclin-Cdk complex.

It is another non-limiting object of the present invention to provide a compound for treating cancer, and which may permit improved treatment for an aggressive cancer associated with Spy1 upregulation and reduced efficacy with treatment by a synthetic Cdk inhibitor.

It is another non-limiting object of the present invention to provide a compound for treating cancer, and which may permit improved resensitization of cancer cells with damaged DNA to cell cycle arrest and apoptosis in the treatment of, for example, breast, brain, liver, blood or ovarian cancer and reduce side effects.

In one aspect, the present invention provides a compound having the following formula or a pharmaceutically acceptable salt, hydrate, solvate, polymorph or prodrug thereof:

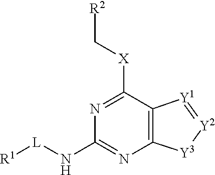

wherein X is O or N; each of Y¹, Y² and Y³ are C or N; L is absent or C₁-C₄ alkylene; R¹ is an optionally substituted aryl or heteroaryl; and R² is an optionally substituted cycloalkyl, bicycloalkyl, tricycloalkyl, cycloheteroalkyl, bicycloheteroalkyl or tricycloheteroalkyl.

In another aspect, the present invention provides a compound having structural formula I, II, III or IV or a pharmaceutically acceptable salt, hydrate, solvate, polymorph or prodrug thereof:

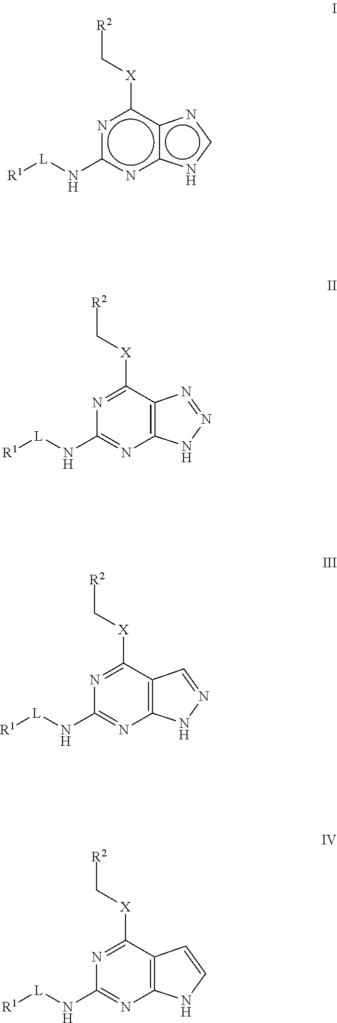

wherein X is O or N; L is absent or C₁-C₄ alkylene; R¹ is an optionally substituted aryl or heteroaryl; and R² is an optionally substituted cycloalkyl, bicycloalkyl, tricycloalkyl, cycloheteroalkyl, bicycloheteroalkyl or tricycloheteroalkyl.

In yet another aspect, the present invention provides a method for treating cancer, the method comprising administering the compound to a patient.

In yet another aspect, the present invention provides a method for identifying, imaging or quantifying a Spy1-Cdk2 complex in a sample, the method comprising contacting the sample with the compound, wherein the compound is coupled to a fluorescent moiety at R¹.

In yet another aspect, the present invention provides a method for identifying a protein target of the compound, the method comprising contacting the compound with a sample, wherein the compound is coupled to a biotin moiety at R¹.

In yet another aspect, the present invention provides a pharmaceutical composition for treating cancer, the composition comprising the compound and a pharmaceutically acceptable excipient.

It is to be appreciated that R¹ is not particularly limited and may include, but not limited to, optionally substituted phenyl, naphthyl, anthracenyl, pyrrolyl, pyrazolyl, pyrazinyl, imidazolyl, triazolyl, acridinyl, benzimidazolyl, benzisoxazolyl, benzofuranyl, benzothiazolyl, benzothiaphenyl, benzoxazolyl, furanyl, indazolyl, indolyl, quinolinyl, oxazolyl, thiazolyl, pyridinyl, thiophenyl or triazinyl. In one embodiment, R¹ is optionally substituted pyrrolyl, furanyl, thiophenyl, imidazolyl, pyrazolyl, oxazolyl, thiazolyl or pyridinyl. In one embodiment, R¹ is optionally substituted phenyl or thiophenyl. In one embodiment, L is methylene, ethylene or propylene.

In one embodiment, L is absent and R¹ is phenyl substituted with one or more of —SO₂NH₂, —SOCH₃ or —CO₂H, or R¹ is

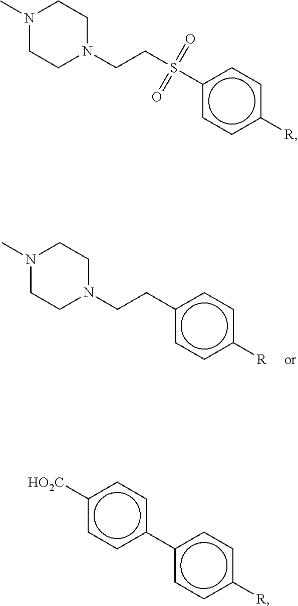

wherein R is remainder of the compound.

In one embodiment, R¹ is

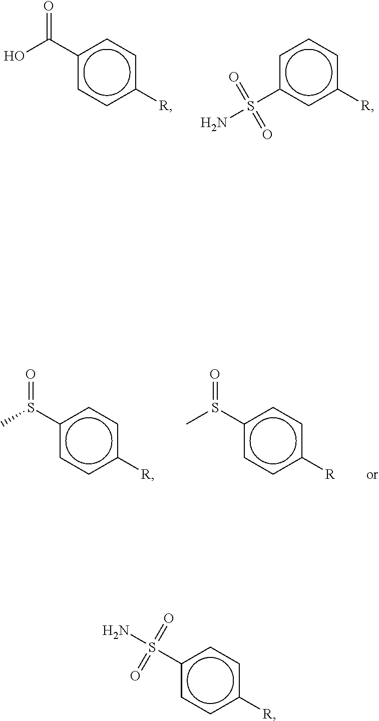

wherein R is the remainder of the compound.

In one embodiment, R¹ is thiophenyl substituted with —SO₂NH₂ or L and R¹ are

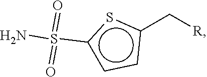

wherein R is remainder of the compound.

In one embodiment, R² is an optionally substituted 5,5-fused, 5,6-fused, 5,7-fused, 6,6-fused, 6,7-fused or 7,7-fused ring system. In one embodiment, R² is an optionally substituted 5,6-fused ring system. In one embodiment, R² is hydrophobic. In one embodiment, R² comprises one or more substituents selected to form a hydrogen bond or a salt bridge in an active site. In one embodiment, R² is an optionally substituted C₅-C₈ cycloalkyl, C₈-C₁₄ bicycloalkyl or 8- to 14-membered bicycloheteroalkyl containing one to four heteroatoms each selected from the group consisting of an oxygen atom, a nitrogen atom and a sulfur atom. In one embodiment, R² is an optionally substituted C₅ or C₆ cycloalkyl, C₈-C₁₀ bicycloalkyl or 8- to 10-membered bicycloheteroalkyl containing one to four heteroatoms each selected from the group consisting of an oxygen atom and a nitrogen atom.

In one embodiment, R² is optionally substituted cyclohexyl, bicyclo[4.3.0]nonyl, bicyclo[3.3.0]octyl, octahydro-1H-indolyl or octahydro-1H-cyclopenta[b]pyridinyl.

In one embodiment, R² is optionally substituted

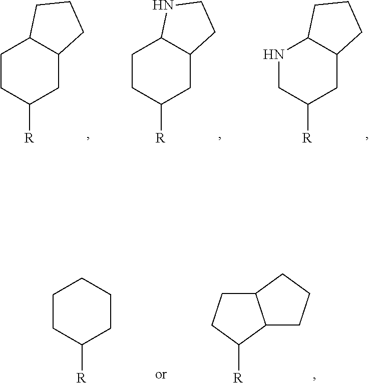

wherein R is remainder of the compound.

In one embodiment, R² is optionally substituted with one or more of —NH₂, —CH₂NH₂, —OH, —CH₂OH, —CO₂H,

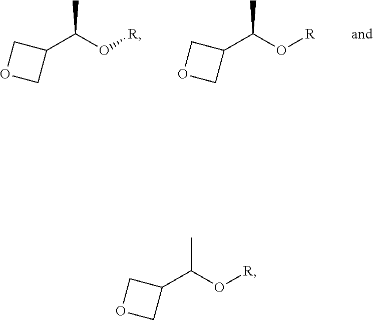

wherein R is remainder of the compound.

In one embodiment, R² is

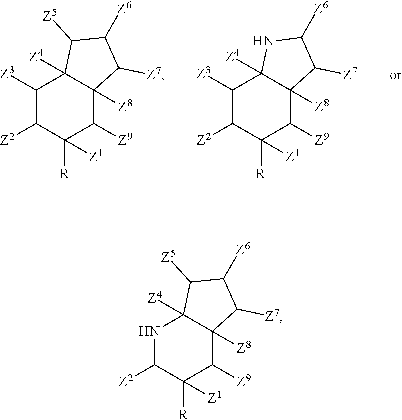

wherein Z1 is —H or —OH; Z2 is —H or

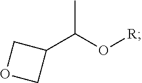

Z³ is —H, —NH₂ or —CH₂NH₂; Z⁴ is —H or —NH₂; Z⁵ is —H, —NH₂ or —CO₂H; Z⁶ is —H, —NH₂, —OH, —CH₂NH₂ or —CH₂OH; Z⁷ is —H, —NH₂, —OH, —CO₂H, —CH₂NH₂ or —CH₂OH; Z⁸ is —H; Z⁹ is —H, —NH₂ or —OH; and R is remainder of the compound.

In one embodiment, R² is

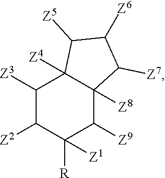

wherein Z¹ and Z² are each —H; Z³ is —H, —NH₂ or —CH₂NH₂; Z⁴ is —H or —NH₂; Z⁵ is —H, —NH₂ or —CO₂H; Z⁶ is —H, —NH₂, —OH, —CH₂NH₂ or —CH₂OH; Z⁷ is —H, —NH₂ or —OH; Z⁸ is —H; Z⁹ is —H or —NH₂; and R is remainder of the compound.

In one embodiment, R² is

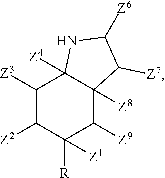

wherein Z¹ is —H or —OH; Z² is —H or

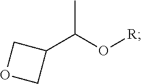

Z³ is —H or —NH₂; Z⁴ is —H; Z⁶ is —H, —CH₂NH₂ or —CH₂OH; Z⁷ is —H, —OH, —NH₂, —CO₂H or —CH₂NH₂; Z⁸ is —H; Z⁹ is —H or —OH; and R is remainder of the compound.

In one embodiment, R² is

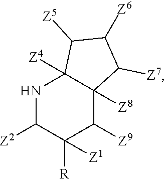

wherein Z¹, Z², Z⁴ to Z⁶, Z⁸ and Z⁹ are each —H; Z⁷ is —H or NH₂; and R is remainder of the compound.

In one embodiment, R² is

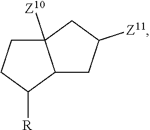

wherein Z¹⁰ is —H or —OH; Z¹¹ is —H or —CH₂NH₂; and R is remainder of the compound.

In one embodiment, R² is

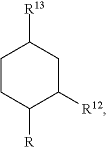

wherein Z¹² is —H or —OH; R¹³ is —H or —CH₂NH₂; and R is remainder of the compound.

In one embodiment, R² is

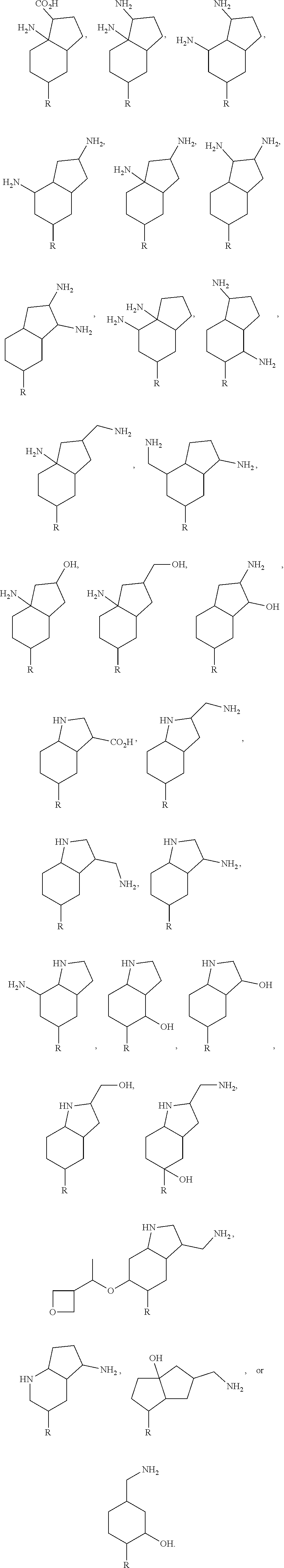

In one embodiment, the compound is one of the compounds listed in Tables 1 and 2 (excluding those in Table 1 “commercially available”, or namely, flavopiridol, dinaciclib, purvalanol A and NU6102). It is to be appreciated that although some compounds are shown in Table 1 with specific stereochemistry, the invention includes all other stereoisomers of the compounds shown in Table 1. Furthermore, while compound I of the invention is shown without any indications of stereochemistry, compound I encompasses all stereoisomers within the scope of structural formula I.

In one embodiment, the compound is coupled to a fluorescent, biotin or proteolysis-targeting chimera (PROTAC) moiety, optionally wherein the fluorescent, biotin or PROTAC moiety is coupled to R¹.

It has been envisioned that the compound may permit operation as a competitive inhibitor for, for example, a cyclin-Cdk complex, or a PROTAC, or coupled to a PROTAC moiety, for inducing intracellular proteolysis.

In one embodiment, the compound is for binding to an active site of a complex comprising a cyclin-dependent kinase and a RINGO/Speedy protein. In one embodiment, the compound is for binding to an active site of a complex comprising a cyclin-dependent kinase and Spy1. In one embodiment, the compound is for binding to an active site of a complex comprising a cyclin-dependent kinase and Spy1, wherein the cyclin-dependent kinase is Cdk1 or Cdk2. In one embodiment, the cyclin-dependent kinase is Cdk2. In one embodiment, the compound is for binding a Cdk1/Cdk2-Spy1 complex to be targeted for degradation. In one embodiment, the compound is for coupling to a fluorescent probe (such as a cyanine, fluorescein, or BoDIPY-derived dye) for imaging, localization and/or quantification of a Spy-Cdk complex. In one embodiment, the compound is for coupling to a biotin moiety to facilitate immunoprecipitation and identification of the Spy1-Cdk interactome.

In one embodiment, the cancer is a breast, brain, liver, blood, prostate, endometrial and ovarian cancer.

In one embodiment, the term “alkyl” refers to a straight-chained or branched hydrocarbon group containing 1 to 12 carbon atoms. The alkyl may include lower alkyl, referring to a C₁-C₆ alkyl chain. Examples of the alkyl group include methyl, ethyl, n-propyl, isopropyl, tert-butyl, and n-pentyl. The Alkyl group may be optionally substituted with one or more substituents.

In one embodiment, the term “alkenyl” refers to an unsaturated hydrocarbon chain that may be a straight chain or branched chain, containing 2 to 12 carbon atoms and at least one carbon-carbon double bond. The Alkenyl group may be optionally substituted with one or more substituents. In one embodiment, the term “alkynyl” refers to an unsaturated hydrocarbon chain that may be a straight chain or branched chain, containing 2 to 12 carbon atoms and at least one carbon-carbon triple bond. The alkynyl groups may be optionally substituted with one or more substituents. The sp² or sp carbons of the alkenyl or alkynyl group may optionally be the point of attachment of the group.

In one embodiment, the term “alkylene” refers to an alkyl group that has two points of attachment, and may preferably include (C₁-C₆) alkylene. In one embodiment, the alkylene is methylene, ethylene, n-propylene or isopropylene.

In one embodiment, the term “amino” refers to a functional group having a nitrogen atom bonded to two hydrogen atoms, where one or both of the hydrogen atoms may optionally be substituted, preferably but not limited to, alkyl or aryl, i.e., the amino includes primary, secondary, tertiary or quaternary amino. For instance, the amino includes alkylamino, dialkylamino or trialkylamino.

In one embodiment, the term “cycloalkyl” refers to a hydrocarbon 3-8 membered monocyclic or 7-14 membered bicyclic ring system having at least one non-aromatic ring. The cycloalkyl group is optionally substituted with one or more substituents, and may be cyclopropyl, cyclopentyl, cyclohexyl, cyclobutyl, cycloheptyl, cyclooctyl, cyclononyl or cyclodecyl.

In one embodiment, the term “heterocycloalkyl” (or “cycloheteroalkyl”) refers to a nonaromatic 5-8 membered monocyclic, 8-12 membered bicyclic or 11-14 membered tricyclic ring system comprising 1-3 heteroatoms if monocyclic, 1-6 heteroatoms if bicyclic or 1-9 heteroatoms if tricyclic, said heteroatoms being O, N, S, B, P or Si. The heterocycloalkyl is optionally substituted with one or more substituents. In one embodiment, the heterocycloalkyl is piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl or thiirene.

In one embodiment, the term “aryl” refers to a hydrocarbon monocyclic, bicyclic or tricyclic aromatic ring system, and which is optionally substituted with one or more substituents. In one embodiment, the aryl is phenyl, naphthyl, anthracenyl, fluorenyl, indenyl or azulenyl.

In one embodiment, the term “aralkyl” refers to aryl attached to another group by a (C₁-C₆)alkylene group. The aralkyl is optionally substituted, either on the aryl portion or the alkylene portion of the aralkyl, with one or more substituent. In one embodiment, the aralkyl is benzyl, 2-phenyl-ethyl or naphth-3-yl-methyl.

In one embodiment, the term “heteroaryl” refers to an aromatic 5-8 membered monocyclic, 8-12 membered bicyclic or 11-14 membered tricyclic ring system having 1-4 ring heteroatoms if monocyclic, 1-6 heteroatoms if bicyclic or 1-9 heteroatoms if tricyclic, where the heteroatoms are independently O, N or S, and the remainder ring atoms are carbon. The heteroaryl is optionally substituted with one or more substituents. In one embodiment, the heteroaryl is pyridyl, 1-oxo-pyridyl, furanyl, benzo[1,3]dioxolyl, benzo[1,4]clioxinyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo[b]thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl or pyrrolo[2,1b]oxazolyl.

In one embodiment, the term “heteroaralkyl” or “heteroarylalkyl” means a heteroaryl group attached to another group by a (C₁-C₆)alkylene. The heteroaralkyl may be optionally substituted, either on the heteroaryl portion or the alkylene portion of the heteroaralkyl, with one or more substituent. In one embodiment, the heteroaralkyl is 2-(pyridin-4-yl)-propyl, 2-(thien-3-yl)-ethyl or imidazol-4-yl-methyl.

In one embodiment, the term “alkoxy” refers to an —O-alkyl radical.

In one embodiment, the term “ester” refers to a —C(O)OR³⁰, wherein R³⁰ is preferably alkyl or aryl.

In one embodiment, the term “halogen” or “halo” is —F, —Cl, —Br or —I. In one embodiment, the term “haloalkyl” is an alkyl group in which one or more hydrogen radicals are replaced by halogen, and may include perhaloalkyl. In one embodiment, the haloalkyl is trifluoromethyl, difluoromethyl, bromomethyl, 1,2-dichloroethyl, 4-iodobutyl or 2-fluoropentyl.

In one embodiment, the term “substituent” or “substituted” means that a hydrogen radical is replaced with a group that does not substantially adversely affect the stability or activity of the compound. The term “substituted” refers to one or more substituents, which may be the same or different, each replacing a hydrogen atom. In one embodiment, the substituent is halogen, hydroxyl, amino, alkylamino, arylamino, dialkylamino, diarylamino, cyano, nitro, mercapto, oxo, carbonyl, thio, imino, formyl, carbamido, carbamyl, carboxyl, thioureido, thiocyanato, sulfoamido, sulfonylalkyl, sulfonylaryl, alkyl, alkenyl, alkoxy, mercaptoalkoxy, aryl, heteroaryl, cyclyl, heterocyclyl, wherein alkyl, alkenyl, alkyloxy, aryl, heteroaryl, cyclyl and heterocyclyl are optionally substituted with alkyl, aryl, heteroaryl, halogen, hydroxyl, amino, mercapto, cyano, nitro, oxo, thioxo or imino.

### BRIEF DESCRIPTION OF THE DRAWINGS

Reference may now be had to the following detailed description taken together with the accompanying drawings in which:

FIG. 1 shows that Spy1 is upregulated in a number of different cancers;

FIG. 2 shows, on top, an activation mechanism for a cyclin A-Cdk2 complex, and on bottom, an activation mechanism for a Spy1-Cdk2 complex;

FIG. 3 shows structure of the Spy1-Cdk complex derived from the crystal structure showing the active site and key topographic features;

FIG. 4 shows, on the left, the binding site of CycA-Cdk2 (A) and on the right, Spy1-Cdk2 (B) occupied by the Cdk2 inhibitor NU-2058 (insert);

FIG. 5 shows, on the left, comparison of calculated docking scores of a library of CKIs binding to Spy1 compared to the canonical complexes, and on the right, overlay of Cdk2 structure when activated by CycA or Spy1, where highlighted are the major structural motifs, specifically the G-loop which undergoes a large structural change resulting in Tyr15 flipping into the active site, and where the image labelled “C” shows electrostatic surface of the active site in CycA-Cdk2, and the image labelled “D” shows electroatatic surface of the Spy1-Cdk2 active site, illustrating the Tyr15 position results in a more closed, non-polar active site;

FIG. 6 shows, on the left, overlay of top two docking poses of NU6102 docked to CycA-Cdk2 (blue) and orientation of the ligand in the crystal structure (yellow) and on the right, alignment of the top docked pose of the NU series of compounds docked to CycA-Cdk2;

FIG. 7 shows binding affinity obtained from MM-GBSA calculations following MD simulations;

FIG. 8 shows comparison of calculated docking scores of currently existing CKIs bound to Spy1-Cdk2 (Blue) and newly designed CKIs and their binding affinities to the Cdk-cyclin complexes (all others);

FIG. 9 shows assessment of proliferation by trypan blue exclusion assay ((n=3); *p<0.05, **p<0.01; ***p<0.001), where 293 cells were transiently transfected with Spy1 or CyclinA and treated with a panel commercially available CKIs;

FIG. 10 shows assessment of proliferation by trypan blue exclusion assay ((n=3); *p<0.05, **p<0.01; ***p<0.001), where A) MDA-MB-231 and B) MDA-MB-468 cells were lentivirally infected with Spy1, CyclinA or CyclinE and treated with a panel of NU series compounds.

FIG. 11 shows assessment of proliferation by trypan blue exclusion assay ((n=3); *p<0.05, **p<0.01; ***p<0.001), where 293 cells were transiently transfected with the panel of Spy1 binding mutants; and

FIG. 12 shows assessment of cell cycle progression ((n=3); *p<0.05, **p<0.01; ***p<0.001) by flow cytometry, where 293 cells were transiently transfected with the panel of Spy1 binding mutants.

### DETAILED DESCRIPTION OF THE PREFERRED EMBODIMENTS

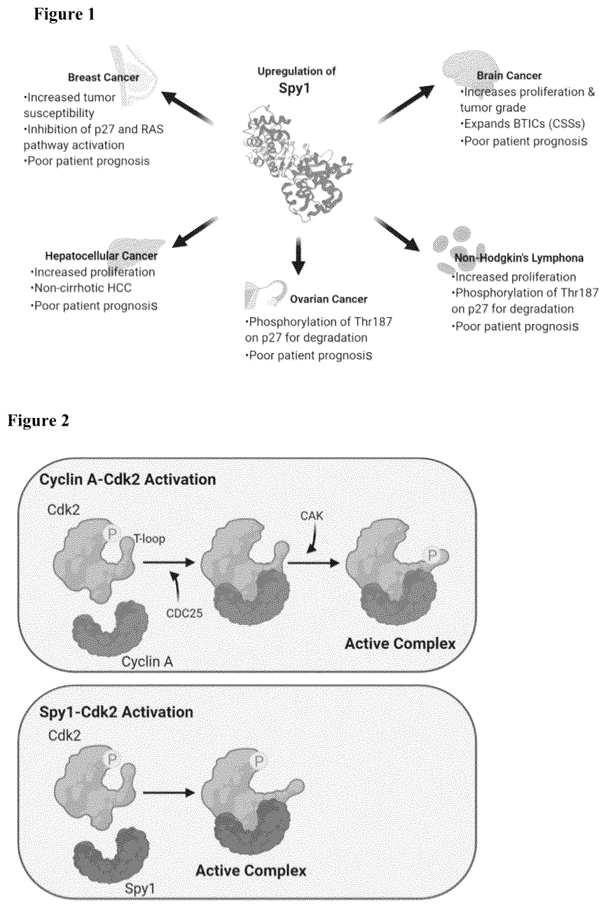

It has been appreciated that one issue not considered by the design and discovery of synthetic CKIs is the existence of Speedy/Ringo, a family of proteins capable of overriding CKI activity, rendering synthetic and natural CKIs less effective. As seen in FIG. 1, it has been also appreciated that Spy1, a member of this family, is elevated in many aggressive cancers, and pre-clinical data in-cells and animals supports that developing drugs to block Spy1 function is a promising therapeutic approach.

Unmet clinical need. Despite increasing survival rates, 30% of women diagnosed with breast cancer will relapse with drug resistant forms of the disease. In addition, approximately 10-20% of all breast cancers initially present as triple negative breast cancer (TNBC), a form that lacks targeted therapy options and, hence, relies on chemotherapy. TNBC affects a younger population of women, has elevated relapse rates and significantly higher mortality rates than other forms of breast cancer. As a result, breast cancer remains the second leading cause of cancer related death among women and represents 13% of annual cancer deaths in Canada. Many other aggressive cancers lack effective targeted therapy options including the most aggressive form of brain cancer, glioblastoma multiforme (GBM). GBM affects patients of all ages ranging from children to the elderly. Average survival is 18 months from diagnosis. While the compound of the invention has been envisioned primarily for aggressive drug resistant forms TNBC and GBM, the target enzyme may also be implicated in the progression of liver, blood, prostate, endometrial and ovarian cancers.

### Scientific and Clinical Relevance of Targeting Cyclin Dependent Kinases (CDKs) in Cancer.

TNBC and GBM share several molecular indicators including elevated protein levels of pro-proliferative proteins, failure of cell checkpoint control and loss of endogenous Cdk inhibitors (CKIs). Hence, it has been appreciated that an attractive targeted therapy may reside with the use of drugs that restore CKI action using synthetic mimics. A first-generation pan-Cdk inhibitor, flavopiridol, has been used in combination with the standard of care Temozolomide (TMZ) in GBM and has shown encouraging results in mice. Despite this, previous synthetic pan-CKI approaches have generally been disappointing showing significant cytotoxic effects, demonstrating insufficient clinical efficacy and target discrimination. Development of second-generation inhibitors have improved targeting to select Cdks and dramatically improved toxicity profiles. The Cdk1/2 inhibitor Dinaciclib has shown promising pre-clinical results in TNBC cells with elevated c-Myc and is in phase II trials. Selective targeting of Cdk2 has not been explored or tested in either GBM or TNBC, but knockout models have shown that Cdk2 is essential for tumorigenesis driven by several classical oncogenes. Cdk4/6 inhibitors, such as palbociclib, have shown promise in GBM but have not shown efficacy in TNBC. Research testing palbociclib in breast cancer has been fundamental for demonstrating the need to stratify patients for CKI treatments. ER+ breast cancer with elevated Cyclin D and reduced p16 levels show significant benefit from palbociclib over hormone therapy alone, improving progression free survival by over 10 months.

### Scientific and Clinical Relevance of Selective Targeting of Spy1-Cdks in Cancers.

Spy1 (SpeedyA, RINGO; gene SPDYA) is a ‘cyclin-like’ protein that directly binds and activates Cdk activity. Spy1 was isolated in a screen for genes that could support proliferation in the face of UV damage. Biochemical data shows that Spy1-activated Cdk1 or Cdk2 are insensitive to senescence-inducing stimuli, including inhibition by endogenous CKIs p21 and p27. It has been appreciated that consequently, Spy1 may represent a novel class of cell cycle regulators capable of inhibiting essential checkpoint mechanisms and actively promoting cell proliferation.

It has been recognized that given the unique mechanism of Spy1, it may represent an attractive hypothesis that inhibition of Spy1 activity could re-sensitize cells with damaged DNA to cell cycle arrest and apoptosis. This could have important clinical impact. Elevated Spy1 protein and gene expression levels are implicated in several cancers including breast, brain, liver, blood and ovarian, and Spy1 levels correlate positively with poor patient outcomes. Targeting Spy1 using lentiviral knockdown reduces cancer proliferation and aggressiveness. Unlike classic cyclins, endogenous Spy1 levels are very low in most somatic cells, except in select stem/progenitor populations where Spy1 drives symmetric division at select times during development. Targeting Spy1 may therefore represent a therapeutic approach with limited side-effects. Our transgenic mouse models with elevated breast and brain Spy1 expression levels show increased cancer rates, aggressiveness and drug resistance. Our PDX zebrafish models show that knockdown of Spy1 in vivo decreases tumour burden and improves response to treatment.

Structural Insights to Spy1 Mechanism of Action. Biochemical data has demonstrated that Spy1 was capable of kinase activation in the absence of required post-translational modification of the Cdk, and in the presence of endogenous CKIs. Resolution of the Spy1-Cdk2 structure revealed differences in Spy1 binding to Cdks that was not apparent from the protein sequence alone. Key interactions between Spy1 and Cdk2 were resolved that lack analogy to any part of the Cyclin-Cdk complex. Spy1 binding altered the conformation of the Cdk to mimic a fully active kinase in the absence of phosphorylation on the Cdk T-loop. Furthermore, resistance to the CKIs p21 and p27 can be explained by the lack of a docking cleft and an MRAIL motif in the Spy1-Cdk2 complex required for CKI binding.

As seen in FIG. 2, in normal healthy adult tissue, CDK2 is activated by Cyclin A and a series of post-translational modifications factors must also come into play. This keeps this protein under tight regulatory control and ensures that cell division is tightly regulated, as seen on top of FIG. 2. However, for Spy1, activation is rapid and requires no additional factors. This means that it can induce cell division, and possibly cancer, without the need for other proteins to become involved. It also renders the regulatory endogenous inhibitors, that can shut down activated CDK2, impotent. Expressing Spy1 is akin to cutting the brakes of a vehicle.

### Desired Mechanism of Modulation and Proposed Modality.

The active site among the Cdk family members has a high degree of homology, which complicates the selectivity of drug targeting. Using molecular modelling, Spy1-Cdk2 and CycA-Cdk2 active sites when bound to various known inhibitors were compared. Surprisingly, it has been shown that distinct differences between the two Cdk-complex active sites exist. Briefly, published crystal structures of the two complexes were used as a starting point. Any ligand (if present) from the binding site was removed and a rigid docking model was used. Induced fit docking was also tested, however rigid docking performed significantly better and was able to consistently reproduce crystal structure ligand poses at <1 Å RMSD. The reason for this being that, while IFD allows for receptor flexibility, this considerably increases computational cost and as a result the thoroughness of the docking/conformational sampling is lower. The sampling of the rigid docking, however, can be significantly increased for only moderate increases in computational cost and this increased sampling yields better results. Each drug was independently docked multiple times; however, in each case 1 primary docking mode dominated for CycA-Cdk2, consistent with reported crystal structures, suggesting the high affinity for these systems. This was not the case with Spy1-Cdk2, indicating that binding was poor. These calculated energies indicate a binding energy preference of 6-9 kcal/mol, translating to approximately a 10⁴ to 10¹ fold preference for CycA for four drugs designed specifically to target the Cdk2 binding pocket exclusive to other kinases, NU-2058, NU-6086, NU-6094, and NU-6102. The lowest energy family of docked structures was then analyzed using MD simulations (20 ns) performed in triplicate with random initial velocities. MD simulations allow for system flexibility. From these calculations, a binding affinity using MM/GBSA was obtained and similarly the ligands showed a preference for CycA-Cdk2 over Spy1-Cdk2, respectively. Existing CKI drugs are essentially ineffective against Spy1-activated Cdk2 but provide a starting point for drug design and synthesis efforts.

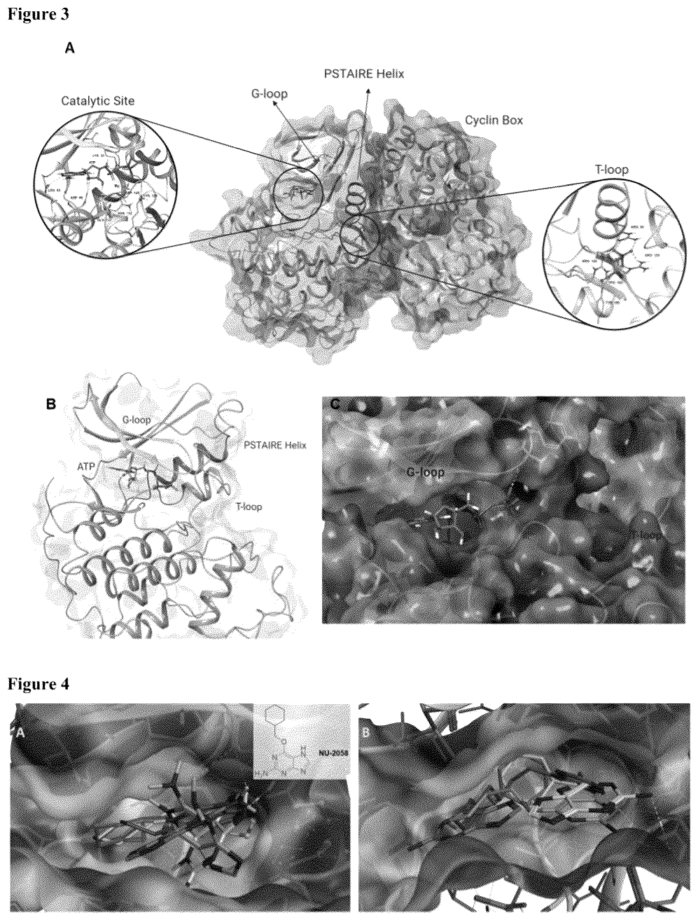

FIG. 3 shows structure of the Spy1-Cdk complex derived from the crystal structure, and which also represents the energy minimized structure that was generated and used for computational drug development. FIG. 4 shows the binding site of the CycA-Cdk2 and Spy1-Cdk2 complexes occupied by NU-2058, where the three lowest energy docking modes are shown for each in purple, green and yellow. In the case of the former complex, the cyclohexyl groups all adopted similar conformations binding the left-hand side of the pocket. The adenosine residues have considerably more flexibility, but all reside on the right-hand side of the pocket and interact with both the enzyme's surface and the water interface. This consistency indicated the preferred geometry and helped explain the good inhibition of the enzyme by this drug. In the case of Spy1, there was much more flexibility. The cyclohexyl groups of two of the conformations bind to the middle of the pocket (yellow and pink) but the adenosines are located on opposite sides indicating poor binding preferences.

### Molecular Modeling

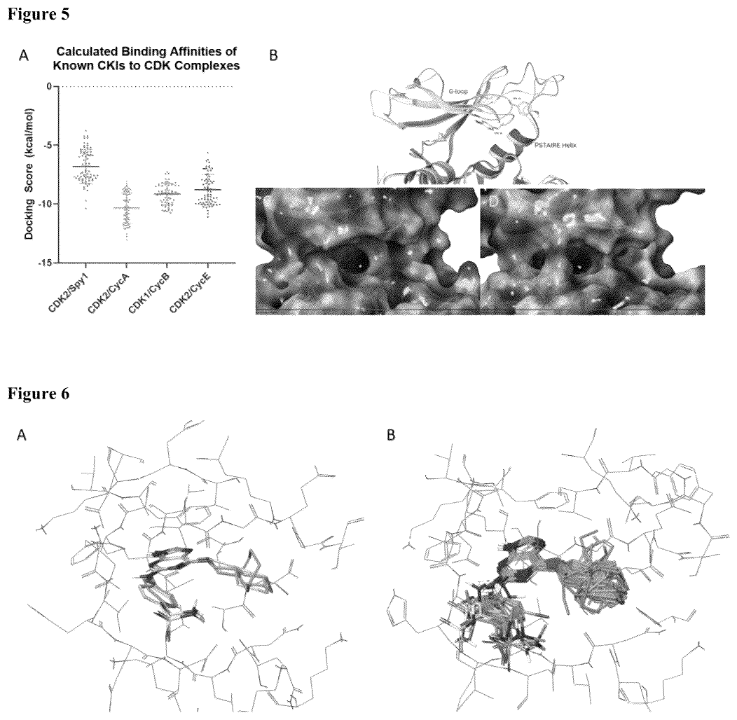

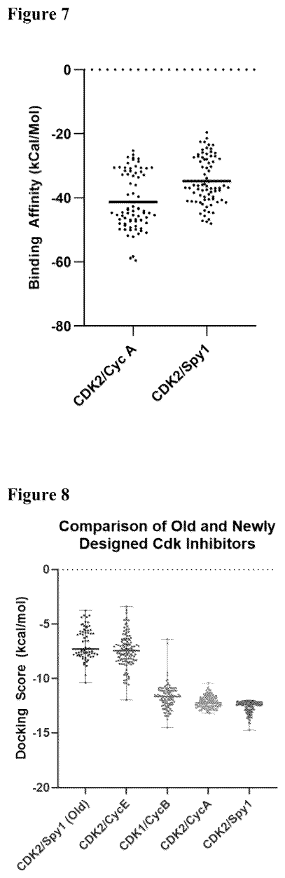

Our docking approach was benchmarked using a library of ˜200 inhibitors binding to the CycA-Cdk2 complex (no binding data is available for Spy1-Cdk2 as it has not been targeted to date). Our investigation explored the ideal crystal structure, rigid receptor and induced fit docking, solvation effects, van der Waal radii and other parameters. Rigid receptor docking was found to give excellent correlation (R²=0.8-0.9 depending on scoring method), and was an excellent balance between computational cost (time) and accuracy and resulting docked structures had <1 Å RMSD when compared to crystal structures (see the CycA-Cdk2 complex in FIG. 6). Docking accuracy was on par with more intensive molecular dynamics/MM-GBSA based approaches which were done for comparison and to account for flexibility of the receptor binding pocket. Several parameters were also tested for MD/MM-GBSA calculations, including partial charge derivation methods, solvation models, and binding affinity method (GBSA vs. PBSA) to obtain optimal results. Computational modelling and comparison of inhibitors binding to the canonical cyclin-Cdk complexes CycA-Cdk2, CycE-Cdk2, and CycB-Cdk1, as well as to our target, Spy1-Cdk2, showed that inhibitors have considerably worse binding to the Spy1-Cdk2 complex (see FIG. 5, left side, and Table 1 below). This result was consistent regardless of the method used to measure binding affinities (see FIG. 5, left side, and FIG. 7). This correlates with the decreased effectiveness observed in the ability of compounds to treat aggressive cancers in which Spy1 is upregulated: they do not bind well enough. Modelling also showed that Cdk2 activation by Spy1 induced a stable conformational change in the active site which results in a smaller, more hydrophobic binding pocket, which hinders the binding of these inhibitors (see FIG. 5, right side).

TABLE 1
Comparison of calculated binding affinities for commercially available and ligands or
compounds of the invention

| | | Docking Scores (kcal/mol) | | | |
|---|---|---|---|---|---|
| | Compound | CDK2/CycA | CDK2/CycE | CDK1/CycB | CDK2/Spy1 |
| Commercially Available | 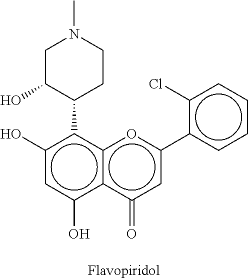 | −9.945 | −9.90 | −10.15 | −8.76 |
| | 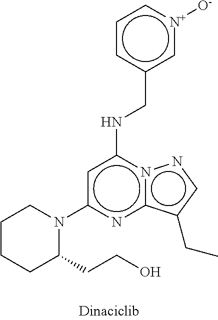 | −12.414 | −11.09 | −8.73 | −8.78 |
| | 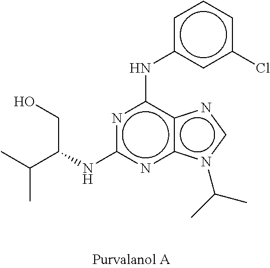 | −10.531 | −9.61 | −8.44 | −6.83 |
| | 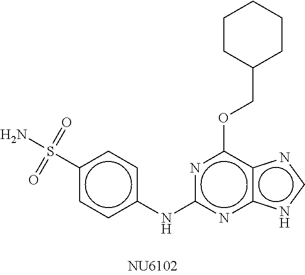 | −12.111 | −9.96 | −11.95 | −8.72 |
| Newly Designed | 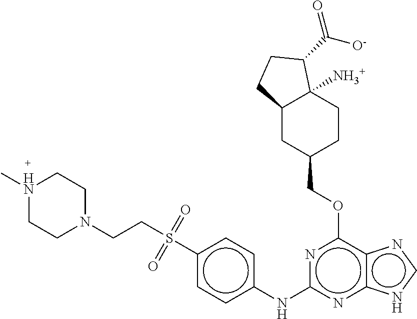 | −12.09 | −6.35 | −11.67 | −14.74 |
| | 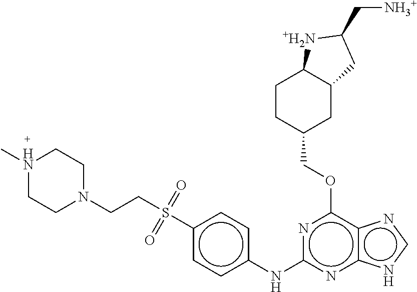 | −11.74 | −6.12 | −12.78 | −14.13 |
| | 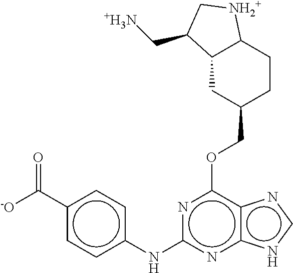 | −14.12 | −5.36 | −11.58 | −14.05 |
| | 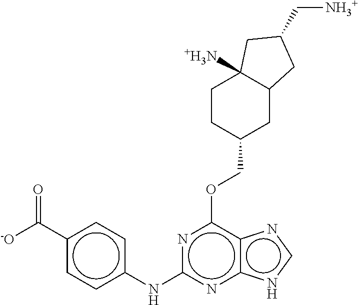 | −10.71 | −6.84 | −11.22 | −14.48 |

Our approach involved computational modelling and screening (using rigid receptor docking followed by molecular dynamics/MM-GBSA to calculate binding affinity) of a large number of inhibitors which have been rationally designed based on the modelling of the target receptor and the properties of the binding pocket. Also utilized was an extensive library screening approach (containing over 2000 pharmaceutically relevant motifs covering most of chemical space) to incorporate functional groups to maximize interactions with the unique properties of the Spy1-Cdk2 active site. Several potential hit compounds (see FIG. 8 and Table 1, above) were identified and predicted to have significantly higher binding affinity to the Spy1-Cdk2 complex, as well as moderate selectivity over the canonical cyclin-Cdk complexes. Generally, more hydrophobic compounds (such as those containing the 6-5 fused rings) are preferred as the inverted tyrosine residue makes the binding site more hydrophobic in that region and sits directly on these rings. Functionalizing these compounds with groups capable of forming hydrogen bonds or salt bridges also significantly improved binding. Compound synthesis may follow an asymmetric, diastereo-divergent approach that features a late-stage incorporation of the key variable moieties, which allows for quick and efficient synthesis of a wide variety of target molecules. The design approach may be iterative, and any data obtained, either computationally or experimentally, may be fed back into the design process to further refine and optimize compounds and their binding affinities. For example, databases of over 100,000 molecules with known kinase activity were computationally screened, and none of these known molecules were predicted to bind better to Spy1 than CycA-activated Cdk2. Additionally, none of the compounds bind Spy1-Cdk2 well, in contrast to the compound of the invention. Over 250,000 other small molecule drugs were also screened to expand the search beyond established CKI based inhibitors as these have consistently shown poor binding. Of these compounds only a handful have shown potential for possible functionalization for further development.

The applicant has appreciated existing challenges with known CKIs include that almost no clinically deployed kinase inhibitor showing much, if any, selectivity. The binding sites are so similar between different kinases, let alone the same kinase activated by a different cyclin. A difference in docking score of 1 kcal/mol suggests a selectivity ratio of approximately 20:1. A difference of 2 kcal/mol is approximately 50:1. It has been envisioned that off-target effects of the compound of the invention may likely to be minimal. While lower selectivity may result in more general Cdk2 inhibition, improved binding to the Spy1 complex may be more desirable. As seen in FIG. 8, compounds of the invention were shown to be on the order of a 1000x better at Spy1 complex binding than any current drug (about 5 kcal/mol, pink vs. blue).

In terms of the synthesis, we have redesigned a route to this class of molecules that greatly improves efficiency, and have made over two dozen model compounds along the lines of the NU series, both known, and new molecules with new functionality and structures.

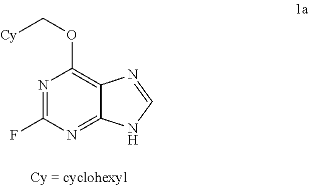

### 6-cyclohexylmethoxy-2-fluoro-purine (1a)—CAUTION, H₂ generation

To a suspension of NaH (60% dispersion in mineral oil; 0.24 g, 6.0 mmol, 3.0 eq) in anhydrous THF (5 mL) under an inert atmosphere (N₂) was added cyclohexylmethanol (0.62 mL, 5.0 mmol, 2.5 eq) dropwise while allowing the H₂ gas generated to escape through a bubbler (Caution! H₂ evolution). The mixture was stirred for 1 h at room temperature, followed by dropwise addition of a solution of 6-chloro-2-fluoropurine (0.345 g, 2.0 mmol, 1.0 eq) in anhydrous THF (8 mL). The reaction was stirred at room temperature for 2 h and then heated at reflux for 1 h until TLC analysis confirmed the reaction as complete (10% MeOH/CH₂Cl₂, Rf=0.53). Water was added (2 mL) and the mixture was neutralized with AcOH. The mixture was concentrated in vacuo and the resulting solid was suspended in MeOH (4 mL) and precipitated using water (30 mL). The precipitate was collected by vacuum filtration and washed with water (2×5 mL) to yield 1a as a white powder (0.373 g, 75%).

¹H NMR (300 MHz, DMSO-d₆) δₚₚₘ 8.38 (s, 1H), 4.34-4.32 (d, J=6.5 Hz, 2H), 1.84-1.64 (m, 6H), 1.32-1.06 (m, 5H). ¹³C NMR (125 MHz, DMSO-d₆) δₚₚₘ 160.4, 157.9, 156.2, 151.4, 143.6, 72.3, 36.7, 29.0, 25.9, 25.1. ¹⁹F NMR (470 MHz, DMSO-d₆) δₚₚₘ 52.0. This reaction was repeated 5 times on scales ranging from 0.3 to 2 g, with yields between 75-79%. Characterization data was consistent with previously reported values.

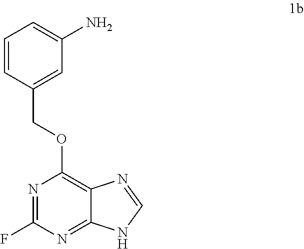 [SMILES: Nc1cccc(COc2nc(F)nc3[nH]cnc23)c1]

### 6-(3-aminobenzyloxy)-2-fluoro-9H-purine (1b)

To a suspension of NaH (60% dispersion in mineral oil; 0.24 g, 6.0 mmol, 3.0 eq) in anhydrous THF (5 mL) under an inert atmosphere (N₂) was added a solution of 3-aminobenzyl alcohol (0.616 g, 5.0 mmol, 2.5 eq) in anhydrous THF (3 mL) dropwise while allowing generated H₂ gas to escape through a bubbler. The mixture was stirred for 1 h at room temperature, followed by dropwise addition of a solution of 6-chloro-2-fluoropurine (0.345 g, 2.0 mmol, 1.0 eq) in anhydrous THF (8 mL). The reaction was stirred at room temperature for 2 h and then heated at reflux for 1 h at which point TLC analysis confirmed the reaction as complete (5% MeOH/EtOAc, Rf=0.33). Water was added (2 mL) and the mixture was neutralized with AcOH. The mixture was concentrated in vacuo and the resulting solid was purified by flash column chromatography (5% MeOH/EtOAc) by dry loading the solid using celite to yield 1b as an off-white solid (0.362 g, 70%).

¹H NMR (300 MHz, DMSO-d₆) δₚₚₘ 8.33 (s, 1H), 7.06-7.00 (apparent t, J=7.5 Hz, 1H), 6.68-6.53 (m, 3H), 5.43 (s, 2H), 5.16 (br s, 2H). ¹³C NMR (125 MHz, DMSO-d₆) δₚₚₘ 161.4, 157.8, 156.2, 148.8, 142.5, 136.1, 129.0, 118.7, 115.8, 114.0, 113.7, 69.3. ¹⁹F NMR (470 MHz, DMSO-d₆) δ ppm 51.9. ESL-MS m/7 calc'd for C₁₂H FN₅O [M+1H]⁺ 260.0948, found 260.0941. This reaction was repeated 2 times on a 0.6 and 1 g scale with yields of 70 and 75%, respectively.

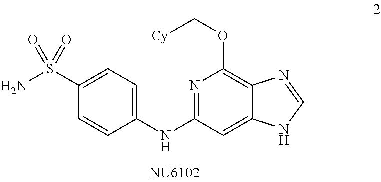

### 4-((6-(cyclohexylmethoxy)-9H-purin-2-yl)amino)benzenesulfonamide (NU6102) (2)

To a solution of 4a (0.10 g, 0.4 mmol, 1.0 eq) in TFE (1 mL) was added TFA (0.15 mL, 2 mmol, 5.0 eq), followed by sulfanilamide (0.138 g, 0.8 mmol, 2.0 eq). The mixture was stirred at reflux for 48 h and then concentrated in vacuo. The residue was diluted with water, washed with saturated NaHCO₃ (2×10 mL), and extracted using EtOAc (3×15 mL). The combined organic layers were dried over MgSO₄ and concentrated in vacuo. The resulting solid was purified by flash column chromatography using a 1-10% MeOH/EtOAc gradient (10% MeOH/EtOAc, Rf=0.57) to yield 2 as an off-white solid (0.127 g, 76%).

¹H NMR (300 MHz, DMSO-d₆) δₚₚₘ 9.76 (s, 1H), 8.03 (s, 1H), 7.97-7.94 (ddd, J=8.7 Hz, 2.1 Hz, 0.7 Hz, 2H), 7.72-7.68 (ddd, J=8.7 Hz, 2.7 Hz, 0.7 Hz, 2H), 7.15 (s, 1H), 4.37-4.35 (d, J=6.0 Hz, 2H), 1.73-1.65 (m, 6H), 1.30-1.18 (m, 5H). Characterization data was consistent with previously reported values.

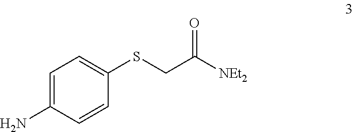

### 2-(4-aminophenyl)sulfanyl-N,N-diethylacetamide (3)

Sodium metal (0.368 g, 15.98 mmol, 1.0 eq) was added to absolute EtOH (40 mL) at room temperature under an inert atmosphere (N₂) and stirred until all of the metal had reacted (Caution! H₂ evolution). 4-Aminothiophenol (2 g, 15.98 mmol, 1.0 eq) was added and the solution was stirred for 15 min. The solution of thiolate was then added dropwise to a solution of N,N-diethylchloroacetamide (2.20 mL, 15.98 mmol, 1.0 eq) in absolute degassed EtOH (40 mL) at 0° C. under an inert atmosphere (N₂). The reaction was stirred for 12 h, water (20 mL) was added and most of the EtOH was removed under reduced pressure. The residual aqueous layer was extracted using CH₂Cl₂ (4×50 mL). The combined organic layers were dried over MgSO₄, and the solvent was removed in vacuo to yield a dark yellow oil. The product was purified by flash column chromatography using (2% MeOH/EtOAc, Rf=0.47) to yield 3 as a dark yellow solid (3.00 g, 78%).

¹H NMR (300 MHz, DMSO-d₆) δₚₚₘ 7.14-7.09 (ddd, J=8.7, 2.7, 2.2, 2H), 6.52-6.47 (ddd, J=8.7, 2.7, 2.1, 2H), 5.27 (s, 2H), 3.55 (s, 2H), 3.28-3.18 (two overlapping q, J=7.2 Hz, 4H), 1.09-1.05 (t, J=7.2 Hz, 3H), 1.01-0.96 (t, J=7.2 Hz, 3H). ¹³C NMR (75 MHz, DMSO-d₆) δₚₚₘ 167.6, 149.31, 134.7, 118.4, 114.7, 42.2, 39.8, 39.0, 14.7, 13.3. ESI-MS m/z cald for C₁₂H₁₉N₂OS [M+H]⁺ 239.1218, found 239.1220. This reaction was repeated 3 times on scales ranging from 1 to 5 g with yields between 78-80%.

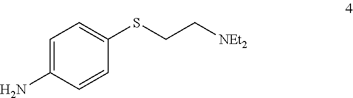

### 4-(2-(diethylamino)ethylsulfanyl)aniline (4) (Procedure 1)

To a suspension of LiAlH₄ (0.280 g, 7.4 mmol, 4.0 eq) in anhydrous THF (15 mL) was added a solution of 3 (0.43 g, 1.85 mmol, 1.0 eq) in THF (5 mL) dropwise with ice cooling (Caution! H₂ may be evolved). The reaction was warmed to room temperature and allowed to stir for 24 h. The reaction was quenched by sequential addition of water (0.25 mL), 15% NaOH solution (0.25 mL), and water (0.75 mL). The mixture was stirred until a white solid had formed, at which point it was filtered through celite followed by washing the celite with ether. The filtrate was dried over MgSO₄ and concentrated in vacuo to yield an oil. The oil was then taken up in CH₂Cl₂ (20 mL) and washed with water (2×10 mL). The organic layer was dried over MgSO₄ and concentrated in vacuo to give 4 as a yellow oil (0.367 g, 90%).

¹H NMR (300 MHz, CD₃OD) δₚₚₘ 7.23-7.18 (ddd, J=8.6 Hz, 2.9 Hz, 1.8 Hz, 2H), 6.68-6.63 (ddd, J=8.6 Hz, 2.9 Hz, 2.0 Hz, 2H), 2.82-2.77 (t, J=8.3 Hz, 2H), 2.66-2.61 (t, J=8.3 Hz, 2H), 2.55-2.48 (q, J=7.2 Hz, 4H), 0.99-0.95 (t, J=7.2 Hz, 6H). This reaction was repeated 2 times on a 0.4 and 2 g scales with yields of 90 and 94%, respectively. Characterization data was consistent with previously reported values.

### (Procedure 2)

Sodium metal (0.386 g, 16.78 mmol, 2.1 eq eq) was added to absolute EtOH (40 mL) at room temperature under an inert atmosphere (N₂) and stirred until all of the metal had reacted (Caution! H₂ evolution). 4-Aminothiophenol (1 g, 7.99 mmol, 1.0 eq) was added and the solution was stirred for 15 min. The solution of thiolate was added dropwise to a solution of 2-chloro-N,N-triethylamine hydrochloride (1.38 g, 7.99 mmol, 1.0 eq) in absolute EtOH (40 mL) at 0° C. under an inert atmosphere (N₂). The reaction was stirred for 24 h at 60° C., water (10 mL) was added and EtOH was removed under reduced pressure. The aqueous layer was extracted using CH₂Cl₂ (4×25 mL). The combined organic layers were dried over MgSO₄, and the solvent was removed in vacuo. The crude product was purified by flash column chromatography (30% MeOH/EtOAc, Rf=0.33) to yield 4 as a yellow oil (0.86 g, 48%). This reaction was performed 2 times on the same scale providing similar yields in both cases (48 and 49%).

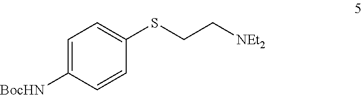

### tert-butyl (4-((2-(diethylamino)ethyl)thio)phenyl)carbamate (5)

To a solution of 4 (0.367 g, 1.64 mmol, 1.0 eq) in THF was added Et₃N (0.46 mL, 3.28 mmol, 2.0 eq) followed by Boc₂O (0.72 g, 3.28 mmol, 2.0 eq). The solution was stirred at reflux for 16 h after which THF was removed under reduced pressure. The residue was taken up in CH₂Cl₂ (15 mL) and washed with 1 M HCl (2×5 mL) and water (10 mL). The organic layer was dried over MgSO₄ and the solvent was removed in vacuo to yield a yellow oil. The product was purified by flash column chromatography (30% MeOH/EtOAc, R_{f}=0.44) to give 5 as a pale-yellow oil (0.49 g, 93%).

¹H NMR (300 MHz, CD₃OD) δₚₚₘ 7.40-7.32 (m, 4H), 3.02-2.96 (t, J=7.8 Hz, 2H), 2.82-2.77 (t, J=7.8 Hz, 2H), 2.73-2.66 (q, J=7.2 Hz, 4H), 1.51 (s, 9H), 1.07-1.03 (t, J=7.5 Hz, 6H). ¹³C NMR (75 MHz, CD₃OD) δₚₚₘ 155.1, 140.1, 133.2, 129.1, 120.3, 81.0, 52.9, 48.1, 31.6, 28.7, 11.0. ESI-MS m/z calc'd for C₇H₂N₂O₂S [M+H]⁺ 325,1950, found 325.1944. This reaction was carried out 2 times on a 0.3 and 1.5 g scale with a yield of 93% both times.

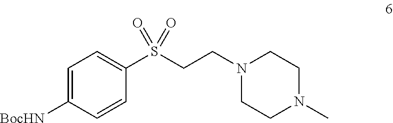 [SMILES: CN1CCN(CCS(=O)(=O)c2ccc(NC(=O)OC(C)(C)C)cc2)CC1]

### tert-butyl (4-((2-(4-methylpiperazin-1-yl)ethyl)sulfonyl)phenyl)carbamate (6)

To a solution of 5 (0.16 g, 0.5 mmol, 1.0 eq) in CH₂Cl₂ (10 mL) was added mCPBA (0.41 g, 2.35 mmol, 4.7 eq) in one portion and the reaction was stirred at room temperature for 3 h. N-methylpiperazine (0.34 mL, 6.0 3.0 mmol, 6.0 eq) was added to the reaction mixture and the reaction was stirred for another 3 h. The reaction mixture was diluted with CH₂Cl₂ (10 mL) and washed with water (2×15 mL) and 5% NaHCO₃ (3×15 mL). The organic layer was dried over MgSO₄ and concentrated in vacuo to yield a pale-yellow oil which was triturated with hexanes to give 6 as a pale-yellow solid (0.17 g, 88%).

¹H NMR (300 MHz, CDCl₃) δₚₚₘ 7.82-7.79 (apparent d, J=8.1 Hz, 2H), 7.56-7.53 (apparent d, J=8.1 Hz, 2H), 6.81 (br s, 1H), 3.28-3.23 (t, J=7.5 Hz, 2H), 2.77-2.72 (t, J=7.5 Hz, 2H), 2.41 (br s, 8H), 2.24 (s, 3H), 1.53 (s, 9H). ¹³C NMR (75 MHz, CDCl₃) δₚₚₘ 152.0, 143.5, 132.9, 129.5, 117.9, 81.8, 54.8, 53.8, 52.6, 51.3, 45.9, 28.3. This reaction was repeated 2 times on a 0.16 and 2 g scale with yields of 88 and 91%, respectively. Characterization data was consistent with previously reported values.

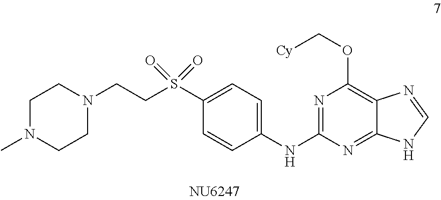

### 6-(cyclohexylmethoxy)-N-(4-((2-(4-methylpiperazin-1-yl)ethyl)sulfonyl)phenyl)-9H-purin-2-amine, (NU6247, 7)

To a solution of 1a (0.050 g, 0.2 mmol, 1.0 eq) in TFE (1 mL) was added 6 (0.154 g, 0.4 mmol, 2.0 eq), followed by TFA (0.075 mL, 1 mmol, 5.0 eq). The reaction was stirred at reflux for 72 h. The mixture is concentrated in vacuo and the residue was diluted with EtOAc (7 mL) and washed with saturated NaHCO₃ (2×4 mL). The organic layer was dried over MgSO₄ and concentrated in vacuo. The resulting solid was purified by flash column chromatography (10% MeOH/CH₂Cl₂, Rf=0.19) to yield 7 as an off-white solid (0.042 g, 41%).

¹H NMR (300 MHz, CDCl₃) δₚₚₘ 7.88 (s, 1H), 7.84 (s, 1H), 7.78-7.75 (apparent d, J=8.7 Hz, 2H), 7.65-7.62 (apparent d, J=8.7 Hz, 2H), 4.28-4.26 (d, J=6.0 Hz, 2H), 3.31-3.26 (t, J=7.1 Hz, 2H), 2.76-2.72 (t, J=7.1 Hz, 2H), 2.38 (br s, 8H), 2.21 (s, 3H), 1.91-1.61 (m, 6H), 1.14-0.81 (m, 5H). ¹³C NMR (125 MHz, CDCl₃) δₚₚₘ 161.0, 154.8, 151.6, 145.5, 140.9, 130.3, 129.9, 117.7, 114.13, 72.6, 54.9, 54.0, 52.8, 51.5, 46.0, 37.4, 29.9, 26.5, 25.8. Characterization data was consistent with previously reported values.

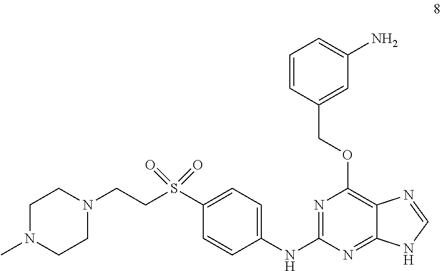 [SMILES: CN1CCN(CCS(=O)(=O)c2ccc(Nc3nc(OCc4cccc(N)c4)c4nc[nH]c4n3)cc2)CC1]

### 6-((3-aminobenzyl)oxy)-N-(4-((2-(4-methylpiperazin-1-yl)ethyl)sulfonyl)phenyl)-9H-purin-2-amine (8)

To a solution of 1b (0.050 g, 0.2 mmol, 1.0 eq) in TFE (1 mL) was added 6 (0.154 g, 0.4 mmol, 2.0 eq), followed by TFA (0.075 mL, 1.0 mmol, 5.0 eq). The reaction was stirred at reflux for 72 h. The mixture was concentrated in vacuo and the residue was diluted with EtOAc (7 mL) and washed with saturated NaHCO₃ (2×4 mL). The organic layer was dried over MgSO₄ and concentrated in vacuo. The resulting solid was purified by flash column chromatography (10% MeOH/CH₂Cl₂, Rf=0.12) to yield 8 as beige solid (0.033 g, 32%).

¹H NMR (500 MHz, CD₃OD) δₚₚₘ 7.99 (s, 1H), 7.63-7.60 (apparent d, J=8.9 Hz, 2H), 7.56-7.54 (apparent d, J=8.9 Hz, 2H), 7.12-7.06 (apparent t, J=8.1 Hz, 1H), 6.86-6.74 (m, 3H), 5.43 (s, 2H), 3.33-3.28 (t, J=7.7 Hz, overlapping with CD₃OD peak, 2H), 2.70-2.65 (t, J=7.7 Hz, 2H), 2.40 (br s, 8H), 2.23 (s, 3H). ¹³C NMR (125 MHz, CD₃OD) δₚₚₘ161.4, 157.8, 156.1, 155.4, 147.9, 141.6, 135.2, 131.1, 128.7, 125.8, 121.4, 114.3, 112.5, 110.6, 110.3, 70.3, 55.5, 54.4, 53.2, 52.6, 45.9. ESI-MS m/z calc'd for C₂₅H₃₁N₈O₃S [M+H]⁺ 523.2240, found 523.2267.

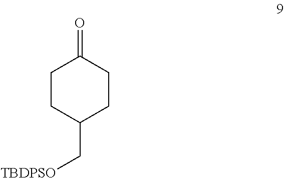

### 4-(((tert-butyldiphenylsilyl)oxy)methyl)cyclohexan-1-one (9)

To a stirred solution of 4-(hydroxymethyl)cyclohexan-1-one (20 g, 128 mmol) and imidazole (11.15 g, 164 mmol) in DCM (400 mL) was drop wise added TBDPSCl (6 mL, 43.7 mmol) and reaction mass was stirred at RT for 16 h. After 16 h, the reaction mass was poured into water (1000 mL) and extracted with DCM (3×600 mL). The combined organics were dried over Na₂SO₄ and concentrated up to 80% volume and reaction mass was kept at RT for 16 h. After 16 h, solid thus obtained was filtered and washed with hexane to give 9 as white crystalline solid (30 g, 81.84 mmol, 52.45%).

¹H NMR (400 MHz, CDCl₃) 7.70 (d, J=7.2 Hz, 4H); 7.50-7.42 (m, 6H), 3.61 (d, J=10 Hz, 2H), 2.46-2.33 (m, 4H), 2.17-2.14 (m, 2H), 2.04-2.03 (m, 1H), 1.55-1.49 (m, 2H), 1.10 (s, 3H).

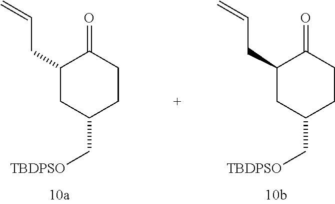

### (±)-(2S,4S)-2-allyl-4-(((tert-butyldiphenylsilyl)oxy)methyl)cyclohexan-1-one (10a) and (±)-(2R,4S)-2-allyl-4-(((tert-butyldiphenylsilyl)oxy)methyl)cyclohexan-1-one (10b)

A stirred solution of diisopropylamine (17.1 mL, 122.95 mmol) in THF (500 mL) was dropwise added 1.6M n-BuLi (77 mL, 122.95 mL) at −78° C. under nitrogen atmosphere and allowed to warm up to 0° C. and stirred at same temperature for 1 h. After 1 h, reaction mass was cooled to −78° C. To this, solution of 4-(((tert-butyldiphenylsilyl)oxy)methyl)cyclohexan-1-one (30 g, 81.84 mmol) in THF (100 mL) was drop wise added and the reaction mass was stirred at same temperature for 1 h. After 1 h, allyl bromide (16.6 mL, 122.95 mmol) was dropwise added at −78° C. and the reaction mass was allowed to stir at RT for 24 h. After 24 h, the reaction mass was poured into water (1000 mL) and extracted with ethyl acetate (3×500 mL). Combined organics were dried over Na₂SO₄ and evaporated to provide crude, which was purified by column purification and product was eluted in 1% ethyl acetate in hexanes to 10a (20 g, 49.18 mmol, 60%) and 10b (4 g, 9.84 mmol, 12%) as clear oils.

10a: ¹H NMR (400 MHz, CDCl₃) 7.67 (d, J=6.8 Hz, 4H); 7.46-7.39 (m, 6H), 5.81-5.74 (m, 1H), 5.06-5.01 (m, 2H), 3.59-3.52 (m, 2H), 2.59-2.55 (m, 1H), 2.46-2.34 (m, 3H), 2.19-2.17 (m, 2H), 2.11-2.07 (m, 1H), 2.01-1.93 (m, 1H), 1.49-1.38 (m, 1H), 1.08 (s, 3H).

10b: ¹H NMR (400 MHz, CDCl₃) 7.68 (d, J=6.8 Hz, 4H); 7.47-7.41 (m, 6H), 5.81-5.72 (m, 1H), 5.08-5.04 (m, 2H), 3.66 (d, J=6.4 Hz, 2H), 2.48-2.40 (m, 2H), 2.36-2.30 (m, 2H), 2.19-2.14 (m, 2H), 2.07-1.99 (m, 1H), 2.1.88-1.81 (m, 2H), 1.75-1.59 (m, 1H), 1.08 (s, 3H).

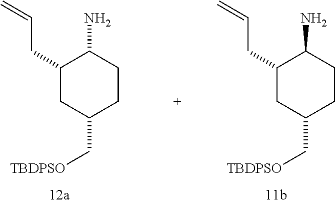

### (±)-(1R,2S,4S)-2-allyl-4-(((tert-butyldiphenylsilyl)oxy)methyl)cyclohexan-1-amine (11a) and (±)-(1S,2S,4S)-2-allyl-4-(((tert-butyldiphenylsilyl)oxy)methyl)cyclohexan-1-amine (11b)

To a stirred solution of 10a (20 g, 49.26 mmol) in methanol (400 mL) was added ammonium acetate (76 g, 986.2 mmol) and sodium cyanoborohydride (15.4 g, 246.3 mmol) in a sealed tube and reaction mass was stirred at 80° C. for 16 h. After completion of the reaction, the reaction mass was concentrated under vacuum; diluted with water (800 mL) and extracted with ethyl acetate (3×400 mL). Combined organics were dried over Na₂SO₄ and evaporated to provide the crude material, which was purified by column chromatography eluting with 1% methanol in DCM using neutral alumina as stationary phase to afford title compounds 11a (7 g, 17.17 mmol, 34.91%) and 11b: (6 g, 14.72 mmol, 29.92%) as yellow liquids.

11a: ¹H NMR (400 MHz, DMSO-d₆) 7.61-7.58 (m, 2H), 7.47-7.43 (m, 6H), 5.82-5.74 (m, 4H), 5.03-4.94 (m, 1H), 3.44 (d, J=6 Hz, 2H), 2.04-1.99 (m, 1H), 1.89-1.82 (m, 2H), 1.64-1.62 (m, 2H), 1.50-1.39 (m, 4H), 1.23-1.14 (m, 1H), 0.99 (s, 9H); LCMS: 408. [M+H]⁺.

11b: ¹H NMR (400 MHz, DMSO-d₆) 7.59-7.57 (m, 4H), 7.49-7.40 (m, 6H), 5.77-5.69 (m, 4H), 5.01-4.96 (m, 1H), 3.46-3.39 (m, 2H), 2.14-2.08 (m, 1H), 1.82-1.66 (m, 6H), 1.47-1.23 (m, 2H), 1.06-1.03 (m, 1H), 0.98 (s, 9H); LCMS: 408. [M+H]⁺.

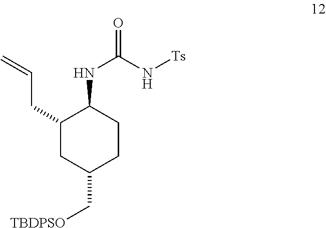

### N-(((1S,2S,4S)-2-allyl-4-(((tert-butyldiphenylsilyl)oxy)methyl)cyclohexyl)carbamoyl)-4-methylbenzenesulfonamide (12)

To a stirred solution of 11b (1.2 g, 2.94 mmol) in DCM (24 mL) was added triethylamine (1.2 mL, 4.99 mmol) and tosyl isocyanate (0.5 mL, 3.24 mmol) at 0° C. and allowed to stir at RT for 16 h. After completion of the reaction, the reaction mass was poured into water (100 mL) and extracted with DCM (3×60 mL). The combined organics were dried over Na₂SO₄ and evaporated to provide crude, which was purified by column purification and product was eluted in 5% methanol in DCM to afford impure product which was further purified by reverse phase column purification using 0.1% formic acid in water: MeCN (0-100%) as a mobile phase to afford 12 as a white solid (0.55 g, 0.91 mmol, 30.89%).

¹H NMR (400 MHz, DMSO-d₆) 8.11 (s, 1H), 7.78 (d, J=8.4 Hz, 2H), 7.66-7.63 (m, 4H), 7.46-7.36 (m, 6H), 7.34-7.27 (m, 2H), 6.36 (d, J=9.2 Hz, 1H), 5.65-5.63 (m, 1H), 4.98 (d, J=8.4 Hz, 1H), 4.91 (d, J=16.8 Hz, 1H), 3.48-3.37 (m, 3H), 2.45 (s, 3H), 2.11-2.05 (m, 1H), 1.96-1.93 (m, 1H), 1.90-1.82 (m, 2H), 1.77-1.73 (m, 1H), 1.56-1.53 (m, 1H), 1.32-1.25 (m, 4H), 1.07 (s, 9H); LCMS: 605.2. [M+H]⁺.

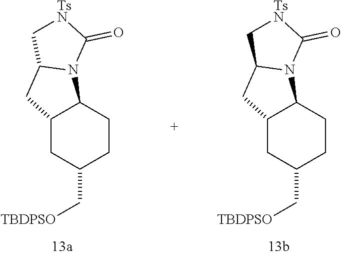

### (4aS,7S,8aR,9aR)-7-(((tert-butyldiphenylsilyl)oxy)methyl)-2-tosyldecahydro-3H-imidazo[1,5-a]indol-3-one (13a) and (4aS,7S,8aR,9aS)-7-(((tert-butyldiphenylsilyl)oxy)methyl)-2-tosyldecahydro-3H-imidazo[1,5-a]indol-3-one (13b)

To a stirred suspension of (diacetoxyiodo)benzene (9.6 g, 29.75 mmol) and palladium(II) acetate (0.083 g, 0.37 mmol) in DCM (50 mL) was dropwise added solution of 12 (4.5 g, 7.44 mmol) in DCM (40 mL) at RT; followed by addition of tetramethylammonium chloride (0.81 g, 7.44 mmol) and sodium acetate (0.61 g, 7.44 mmol). The reaction mass was stirred at RT for 24 h. After completion of the reaction, the reaction mass was concentrated to 0.2V by downward distillation at 50-55° C. to provide crude, which was purified by column purification and product was eluted in 1% methanol in DCM to afford two isomers, 13a (0.6 g, 0.99 mmol, 13.38%) as a light yellow solid and impure 13b (1 g, LCMS: 39%, 0.64 mmol, 8.70%) as a brown semi-solid. LCMS: 603.2. [M+H]⁺.

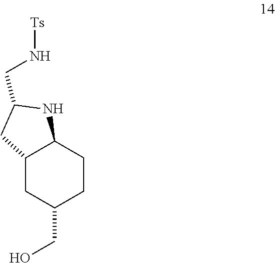

### N-(((2R,3aR,5S,7aS)-5-(hydroxymethyl)octahydro-1H-indol-2-yl)methyl)-4-methylbenzenesulfonamide (14)

To a stirred solution of 13a (0.3 g, 0.497 mmol) in acetic acid (15 mL) was added conc. HCl (15 mL) and stirred in microwave at 140° C. for 1 h. After completion of the reaction, the reaction mass was poured into crushed ice and basified with 2 N NaOH solution (to pH 9); extracted with DCM (3×50 mL). Combined organics were dried over Na₂SO₄ and concentrated by downward distillation (50-55° C.) to provide crude, which was dissolved in methanol (10 mL) and NaOH (60 mg, 1.49 mmol) was added, and reaction mass was stirred at RT for 3 h. After 3 h, reaction mass was concentrated by downward distillation of methanol to afford crude 14 (0.15 g, 0.4 mmol, 81.8%) as a brown semi-solid. LCMS: 339.2. [M+H]⁺.

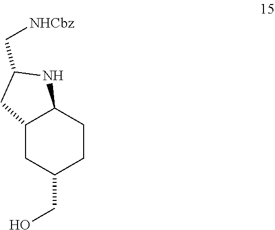

### Benzyl (2R,3aR,5S,7aS)-2-((((benzyloxy)carbonyl)amino)methyl)-5-(hydroxymethyl)octahydro-1H-indole-1-carboxylate (15)

To a stirred solution of 14 (0.15 g, 0.81 mmol) in THF (15 mL) was added liquid ammonia (generated from a balloon of ammonia via cold funnel) at −78° C. After 5 min, sodium metal (0.65 g, 16.3 mmol) was added to it and stirred at the same temperature for 30 min. After completion of the reaction, the reaction mixture was warmed up to RT and 2 M NaOH solution (2 mL) was added, followed by addition of benzyl chloroformate (0.4 mL, 2.44 mmol) and the reaction mass was stirred at RT for 16 h. After completion of the reaction, the reaction mass was poured into water (10 mL) and extracted with DCM (3×20 mL). The combined organics were dried over Na₂SO₄ and solvent was evaporated by downward distillation (50-55° C.) to provide the crude product, which was purified by reverse phase column purification and product was eluted in 80% MeCN in water to afford 15 (0.04 g, 0.088 mmol, 19.94%) as a light yellow solid.

¹H NMR (400 MHz, CDCl₃): 7.37 (m, 10H), 5.81 (bs, 1H), 5.18-5.10 (m, 4H), 4.08-3.98 (m, 1H), 3.72-3.58 (m, 1H), 3.56-3.31 (m, 4H), 2.98-2.60 (m, 2H), 2.18-2.05 (m, 1H), 1.98-1.79 (m, 2H), 1.49-1.28 (m, 3H), 1.03-0.85 (m, 2H); LCMS: 453.3. [M+H]⁺.

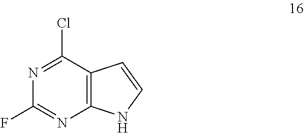 [SMILES: Fc1nc(Cl)c2cc[nH]c2n1]

### 4-chloro-2-fluoro-7H-pyrrolo[2,3-d]pyrimidine (16)

In a plastic tube, 4-chloro-7H-pyrrolo[2,3-d]pyrimidin-2-amine (4 g, 23.72 mmol) was cooled to −60° C. To this was added 70% HF in pyridine (80 mL) and tert-butyl nitrite (7.3 g, 71.17 mmol) dropwise at the same temperature and the reaction mass was stirred at −60° C. for 2 h. After completion of the reaction, reaction mass was poured into ice-cold sat. aqueous NaHCO₃ (1000 mL) and extracted with ethyl acetate (3×250 mL). The combined organic layers were dried over anhydrous Na₂SO₄ and concentrated under vacuum to afford the crude. The crude was purified by column chromatography and the product was eluted with 20% EtOAc/hexanes to afford 16 (2 g, 11.66 mmol, 49.13%) as an off-white solid.

¹H NMR (400 MHz, CDCl₃) δ 9.39 (s, 1H), 7.38-7.30 (m, 1H), 6.70 (dd, J=3.8, 2.0 Hz, 1H). LCMS [M+H]⁺=172.

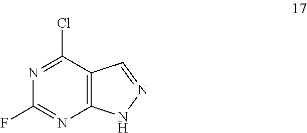 [SMILES: Fc1nc(Cl)c2cn[nH]c2n1]

### 4-chloro-6-fluoro-1H-pyrazolo[3,4-d]pyrimidine (17)

To a cooled solution of 4-chloro-1H-pyrazolo[3,4-d]pyrimidin-6-amine (4 g, 23.6 mmol) in HBF₄ (48% in water, 80 mL) was added a solution of NaNO₂ (3.2 g, 47.3 mmol) in water (40 mL) dropwise at 0° C. and the reaction mass was stirred at RT for 1 h. After completion of the reaction, the reaction mass was neutralized with 6 M NaOH and extracted with EtOAc (3×150 mL). The combined organic layers were dried over anhydrous Na₂SO₄ and concentrated under vacuum to afford 17 (2 g, 11.5 mmol, 49.2%) as a yellow solid.

¹H NMR (400 MHz, DMSO-d⁶) δ 8.54 (s, 1H), 14.60 (s, 1H). LCMS: [M+H]⁺=172.55.

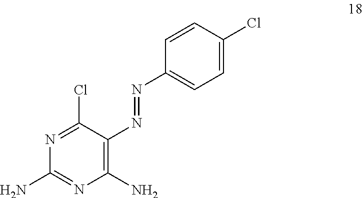 [SMILES: Nc1nc(N)c(/N=N/c2ccc(Cl)cc2)c(Cl)n1]

### (E)-6-chloro-5-((4-chlorophenyl)diazenyl)pyrimidine-2,4-diamine (18)

To a solution of 4-chloroaniline (15 g, 114.14 mmol) in water (105 mL) was added conc. HCl (33 mL) and the reaction mass was stirred for 10 minutes. To this, a solution of NaNO₂ (8.4 g, 120.2 mmol) in water (105 mL) was added dropwise at 0° C., which resulted in a clear solution. In a separate flask, acetic acid (525 mL) and NaOAc (205 g, 2488.8 mmol) were added to a suspension of 6-chloro pyrimidine-2,4-diamine (15 g, 103.7 mmol) in water (525 mL). To this, the above prepared clear solution was added dropwise to the suspension at room temperature and the reaction mass was stirred at room temperature for 16 h. After completion of reaction, the reaction mass was filtered and washed with water (500 mL) and dried under vacuum to afford 18 (28 g, 98.9 mmol, 95.31%) as a yellow solid.

¹H NMR (400 MHz, DMSO-d⁶) δ 9.25 (s, 1H), 8.18 (s, 1H), 7.82 (d, J=8.5 Hz, 2H), 7.57 (d, J=8.5 Hz, 2H), 7.36 (s, 1H). LCMS: [M+H]⁺=283.12.

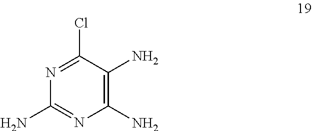

### 6-chloropyrimidine-2,4,5-triamine (19)

To a stirred solution of 18 (28 g, 98.9 mmol) in THF (30V), acetic acid (5V) and Zn dust (45 g, 692.5 mmol) were added at 0° C. The reaction mass was stirred at same temperature for 2 h. After completion of reaction, the reaction mass filtered through celite pad. The filtrate was concentrate under vacuum to get crude. The crude was purified by column chromatography and the product was eluted in 10% methanol in DCM to afford 19 (10.5 g, 65.80 mmol, 66.53%) as a brown solid. LCMS: [M+H]⁺=159.58.

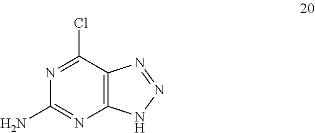 [SMILES: Nc1nc(Cl)c2nn[nH]c2n1]

### 7-chloro-3H-[1,2,3]triazolo[4,5-d]pyrimidin-5-amine (20)

To a stirred solution of 19 (10 g, 62.6 mmol) in water (90V), acetic acid (20V) and sodium nitrite (5.2 g, 75.2 mmol) were added at 0° C. The reaction mass was stirred at same temperature for 2 h. After completion of reaction, the reaction mass filtered through celite pad. The filtrate was concentrate under vacuum to afford 20 (4.1 g, 24.04 mmol, 38.36%) as a brown solid. LCMS: [M+H]⁺=170.56.

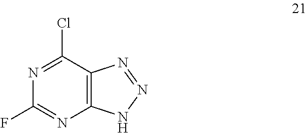 [SMILES: Fc1nc(Cl)c2nn[nH]c2n1]

### 7-chloro-5-fluoro-3H-[1,2,3]triazolo[4,5-d]pyrimidine (21)

To a cooled solution of 20 (4 g, 23.6 mmol) in HBF₄ (48% in water, 80 mL) and cooled to 0° C. To this, a solution of sodium nitrate (3.2 g, 47.3 mmol) in water (40 mL) were added dropwise at the 0° C. and the reaction mass was stirred at RT for 1 h. After completion of reaction, the reaction mass was filtered and washed with water, and the filtrate was extracted with DCM (6×20 mL). The combined organic layer was dried over anhydrous Na₂SO₄ and concentrated under vacuum at 25° C. followed by prep HPLC purification to afford the 21 (0.17 g, 0.979 mmol, 5.57%) as a yellow solid. ¹H NMR (400 MHz, DMSO-d⁶) δ 13.6 (bs, 1H). LCMS: [M−H]⁺=172.55.

### Cell Based Screening Assays

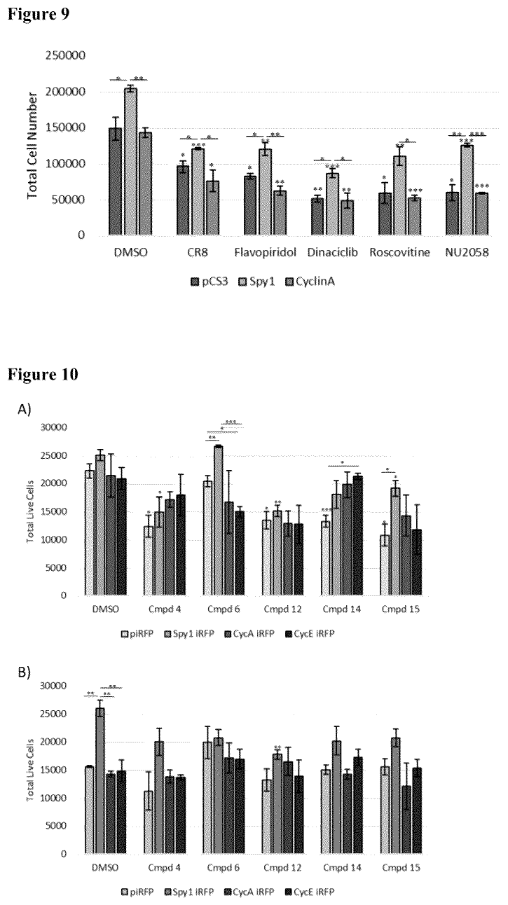

As seen in FIGS. 9 and 10, in vitro cell line work was completed testing commercially available CKIs and NU series compounds to compare Cyclin A/E vs. Spy1 overexpression to compare experimental data with computational modeling of the same CKIs. This work confirmed molecular modeling, showing increased binding to CycA-Cdk2.

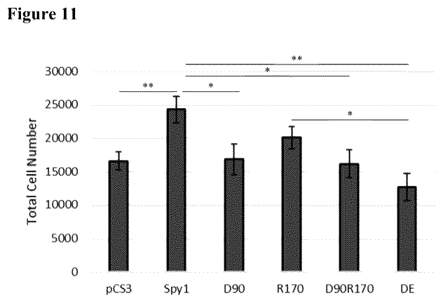

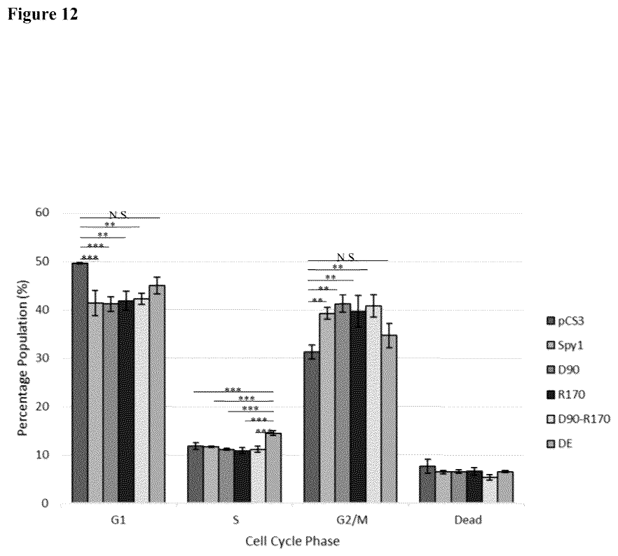

To address the mechanism of action for the compounds which abrogate binding, as seen in FIGS. 11 and 12, in vitro cell work was completed on a panel of Spy1 binding mutants to assess overall rates of proliferation and cell cycle profile analysis to determine if abrogating Spy1-Cdk binding impairs proliferation.

Listed below as Table 2 are preferred non-limiting compounds within the scope of the invention and docketing scores for Spy1-Cdk2 and CycA-Cdk2 complexes.

TABLE 2

| | | Docking Score | Docking Score |
|---|---|---|---|
| Compound | Structure | CDK2/Spy1 | CDK2/CycA |
| 1 | 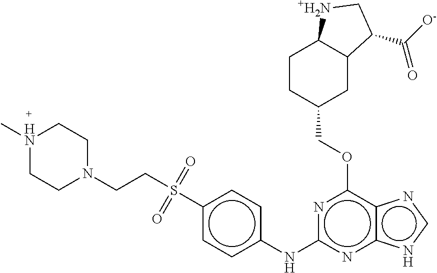 | −14.97 | −11.859 |
| 2 | 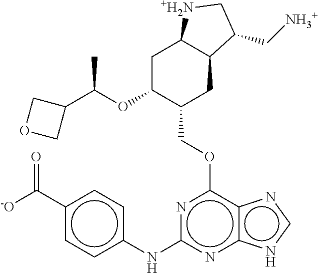 | −14.747 | −16.452 |
| 3 | 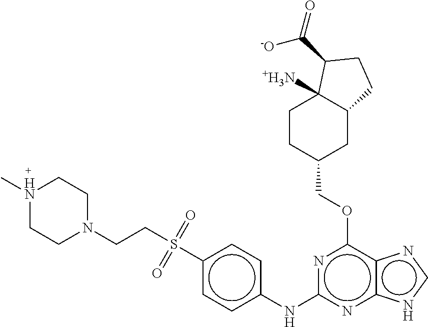 | −14.744 | −12.723 |
| 4 | 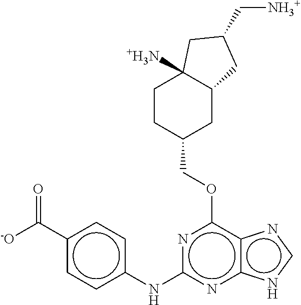 | −14.484 | −15.202 |
| 5 | 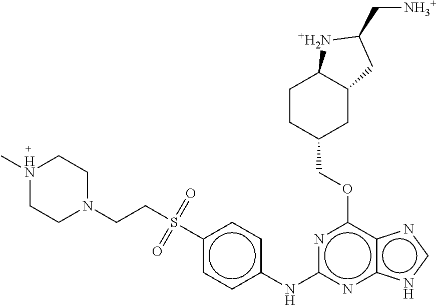 | −14.131 | −12.802 |
| 6 | 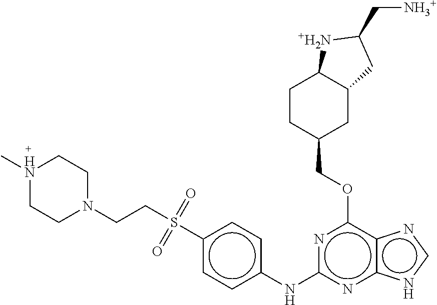 | −14.075 | −12.589 |
| 7 | 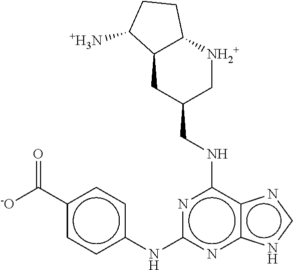 | −13.879 | −11.18 |
| 8 | 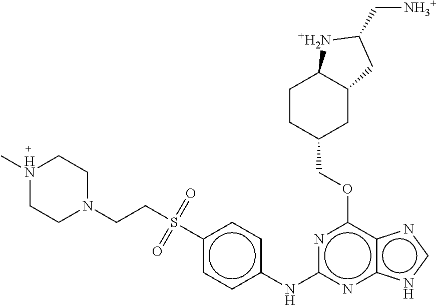 | −13.858 | −12.419 |
| 9 | 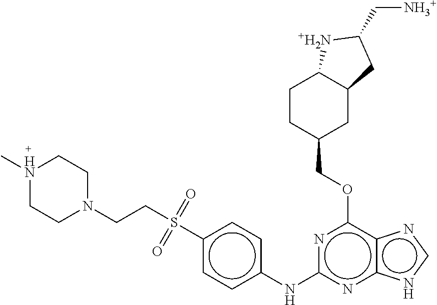 | −13.735 | −12.698 |
| 10 | 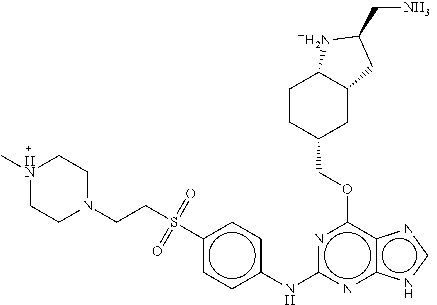 | −13.64 | −12.808 |
| 11 | 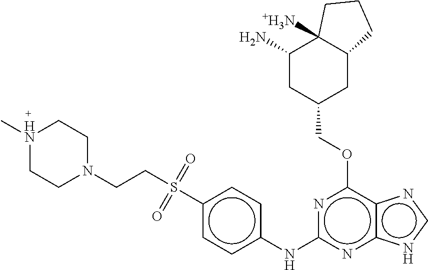 | −13.589 | −13.608 |
| 12 | 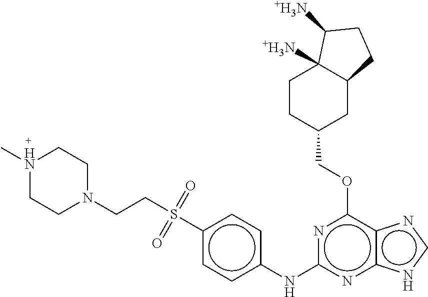 | −13.578 | −12.805 |
| 13 | 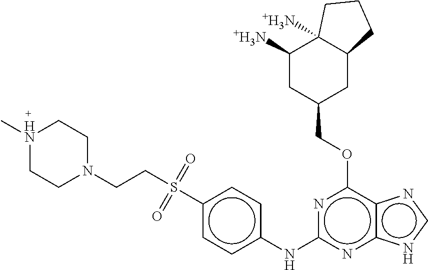 | −13.575 | −12.838 |
| 14 | 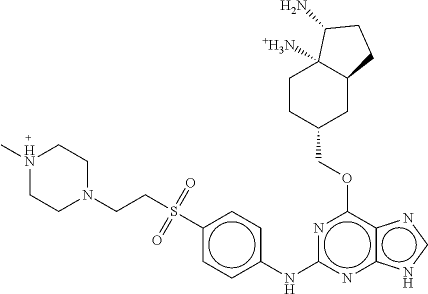 | −13.481 | −12.183 |
| 15 | 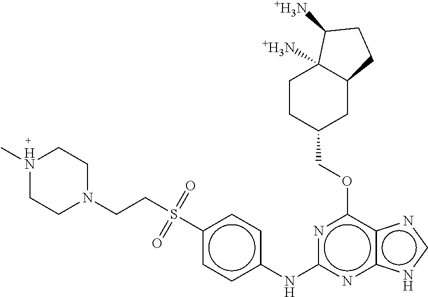 | −13.449 | −12.724 |
| 16 | 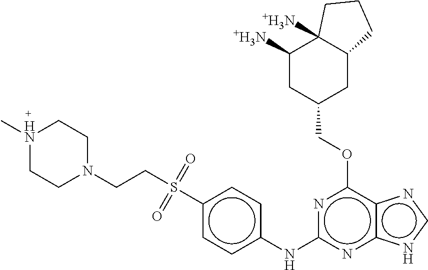 | −13.389 | −12.989 |
| 17 | 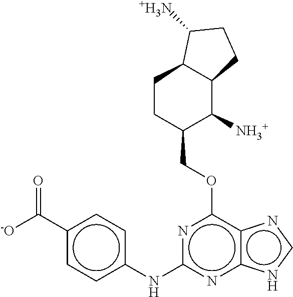 | −13.38 | −13.543 |
| 18 | 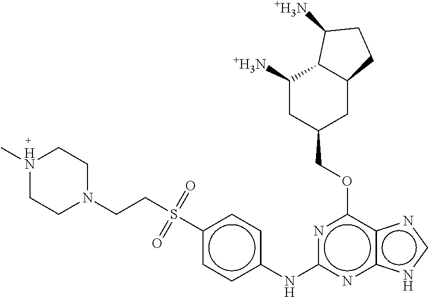 | −13.358 | −12.649 |
| 19 | 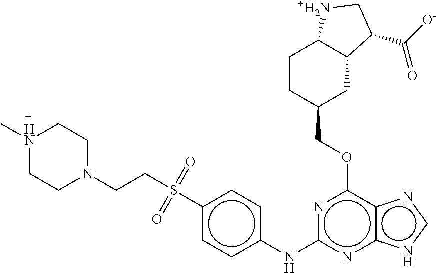 | −13.316 | −12.2 |
| 20 | 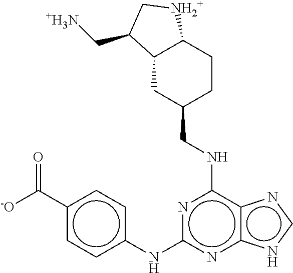 | −13.304 | −10.88 |
| 21 | 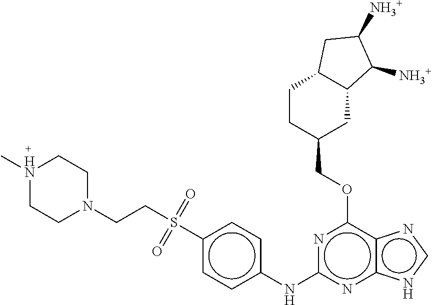 | −13.264 | −13.134 |
| 22 | 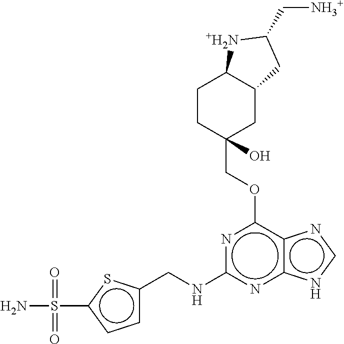 | −13.259 | −12.245 |
| 23 | 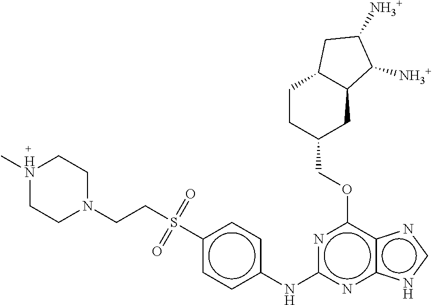 | −13.243 | −12.486 |
| 24 | 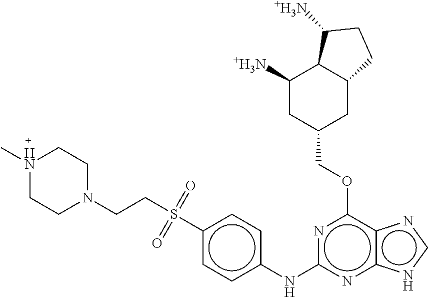 | −13.233 | −13.314 |
| 25 | 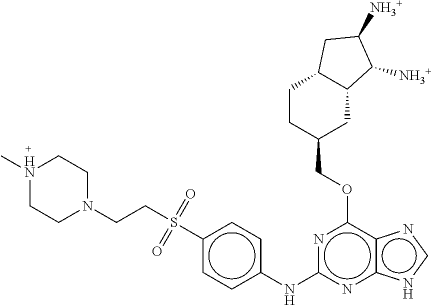 | −13.228 | −13.263 |
| 26 | 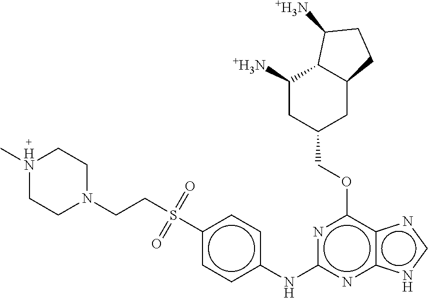 | −13.212 | −12.638 |
| 27 | 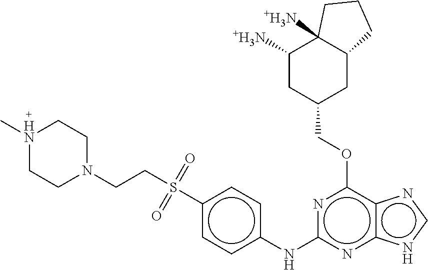 | −13.165 | −12.805 |
| 28 | 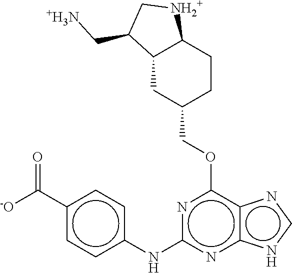 | −13.143 | −12.546 |
| 29 | 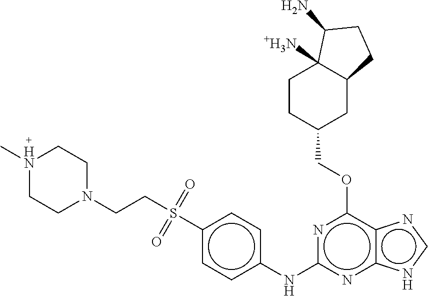 | −13.131 | −12.244 |
| 30 | 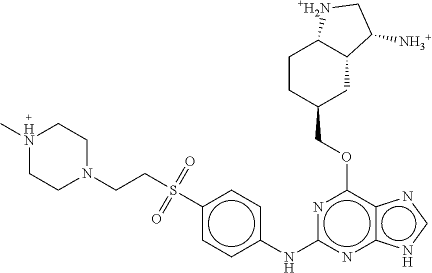 | −13.115 | −13.071 |
| 31 | 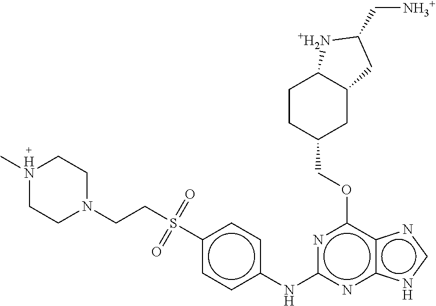 | −13.104 | −12.793 |
| 32 | 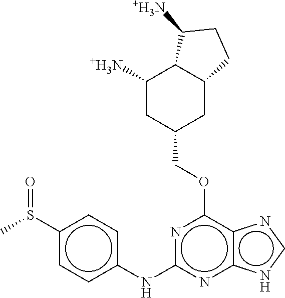 | −13.1 | −12.974 |
| 33 | 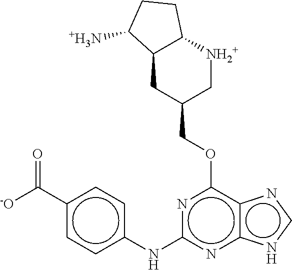 | −13.086 | −11.294 |
| 34 | 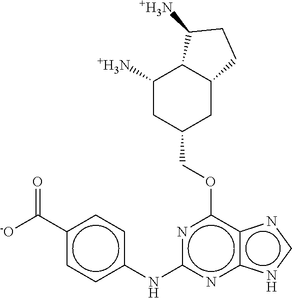 | −13.083 | −16.389 |
| 35 | 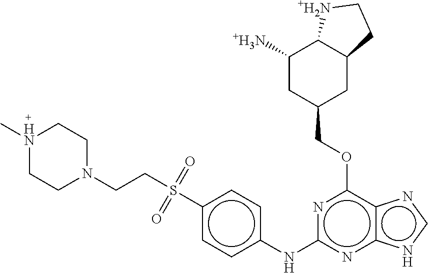 | −13.071 | −12.534 |
| 36 | 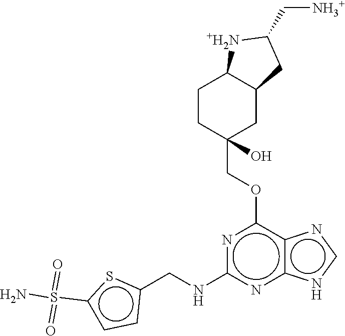 | −13.06 | −12.759 |
| 37 | 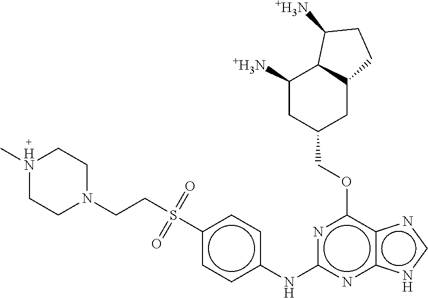 | −12.999 | −13.42 |
| 38 | 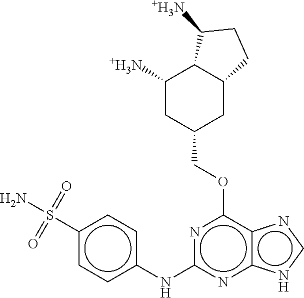 | −12.996 | −12.857 |
| 39 | 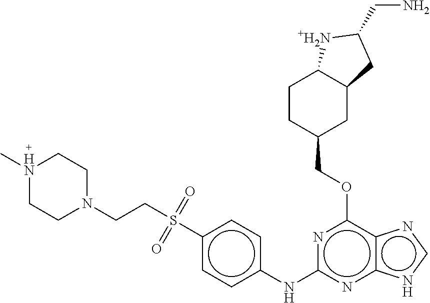 | −12.988 | −12.56 |
| 40 | 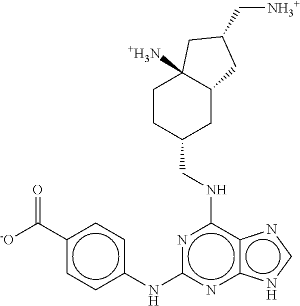 | −12.967 | −10.471 |
| 41 | 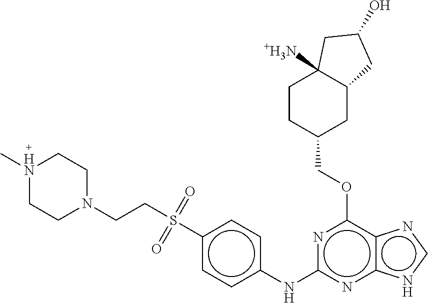 | −12.965 | −12.399 |
| 42 | 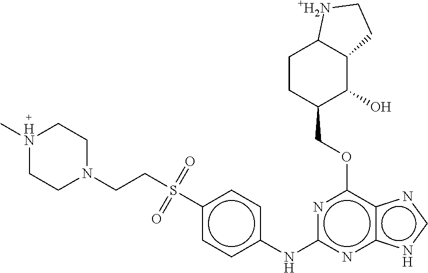 | −12.944 | −12.575 |
| 43 | 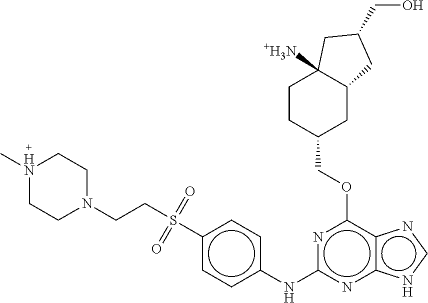 | −12.933 | −12.89 |
| 44 | 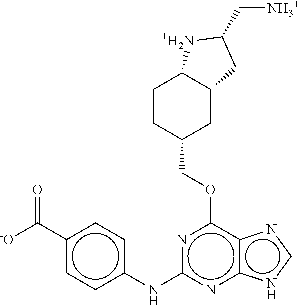 | −12.918 | −15.708 |
| 45 | 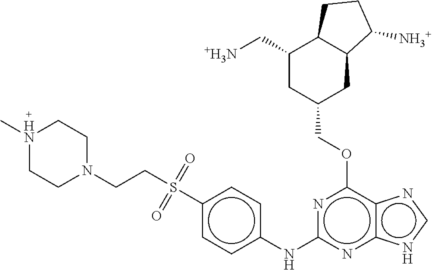 | −12.899 | −11.915 |
| 46 | 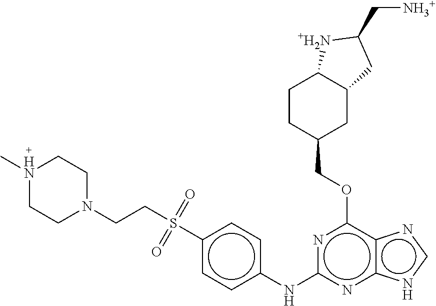 | −12.88 | −12.144 |
| 47 | 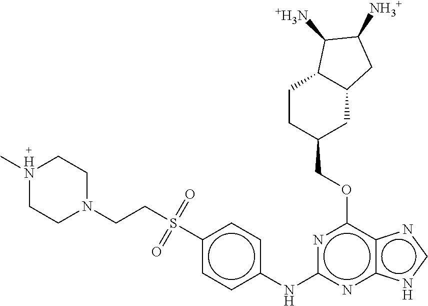 | −12.878 | −12.319 |
| 48 | 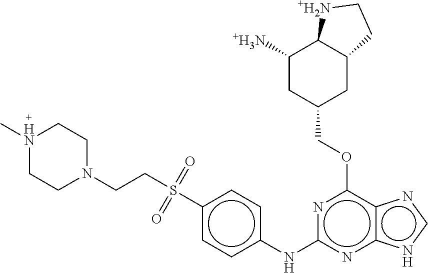 | −12.875 | −12.707 |
| 49 | 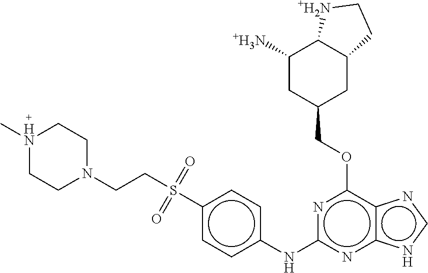 | −12.871 | −12.083 |
| 50 | 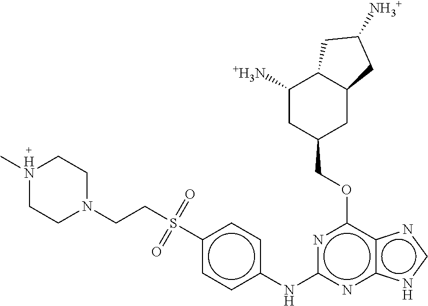 | −12.868 | −12.433 |
| 51 | 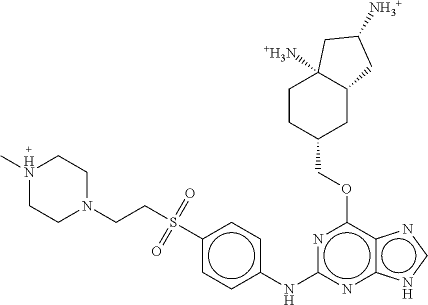 | −12.868 | −13.097 |
| 52 | 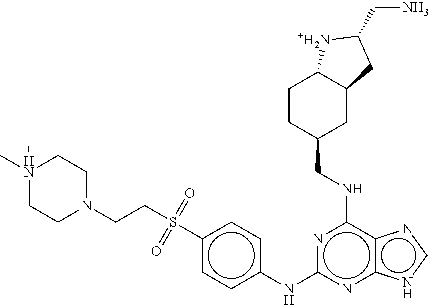 | −12.866 | −12.353 |
| 53 | 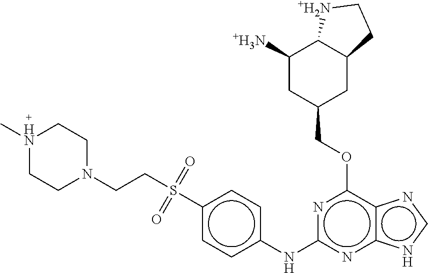 | −12.861 | −12.226 |
| 54 | 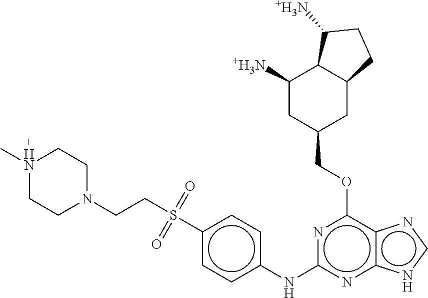 | −12.857 | −12.62 |
| 55 | 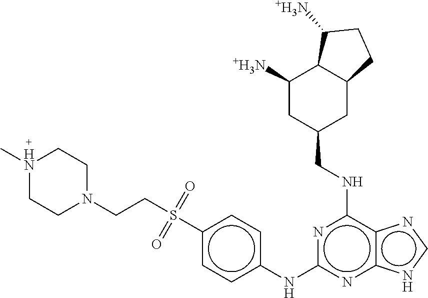 | −12.853 | −11.989 |
| 56 | 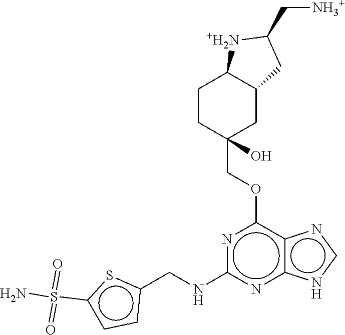 | −12.845 | −11.482 |
| 57 | 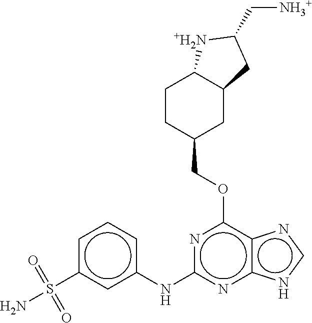 | −12.829 | −12.681 |
| 58 | 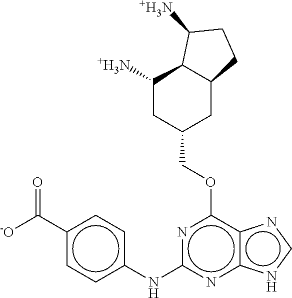 | −12.827 | −15.649 |
| 59 | 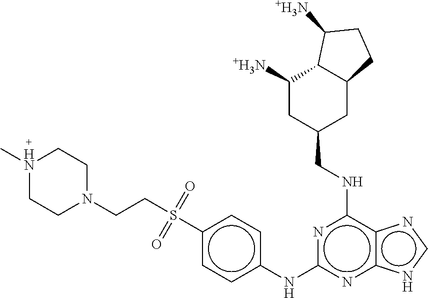 | −12.82 | −12.049 |
| 60 | 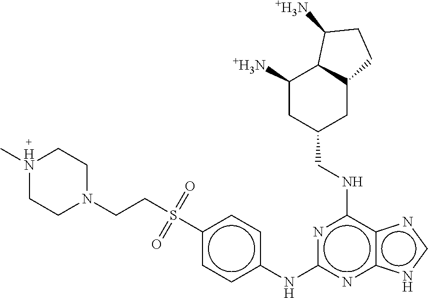 | −12.782 | −12.19 |
| 61 | 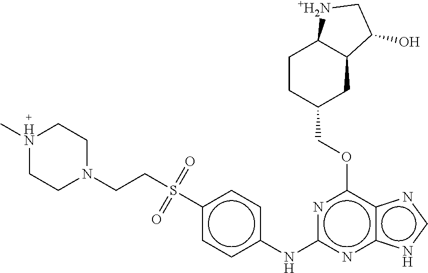 | −12.774 | −11.619 |
| 62 | 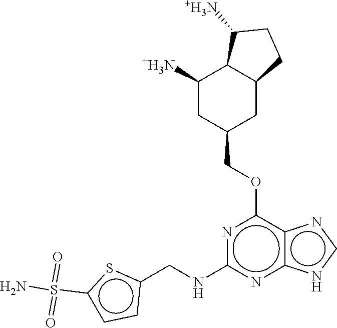 | −12.744 | −10.405 |
| 63 | 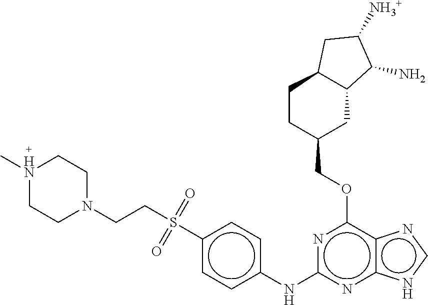 | −12.715 | −12.926 |
| 64 | 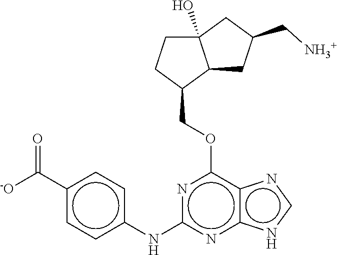 | −12.673 | −14.946 |
| 65 | 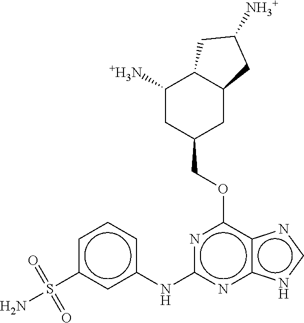 | −12.659 | −12.8 |
| 66 | 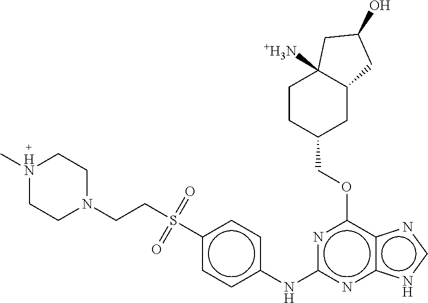 | −12.643 | −12.026 |
| 67 | 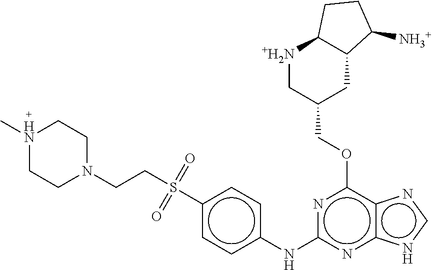 | −12.637 | −12.314 |
| 68 | 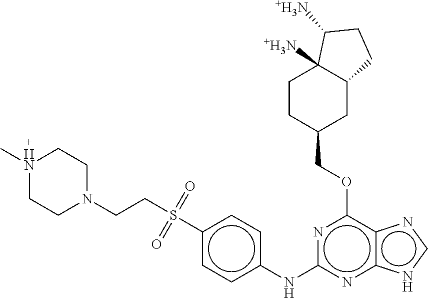 | −12.612 | −13.08 |
| 69 | 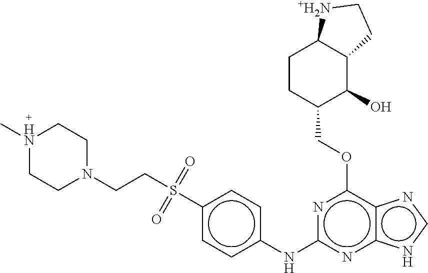 | −12.591 | −12.247 |
| 70 | 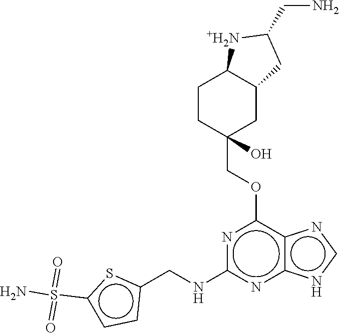 | −12.583 | −12.363 |
| 71 | 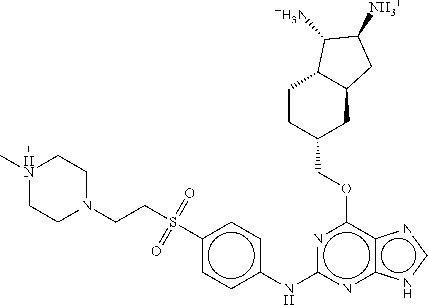 | −12.564 | −12.562 |
| 72 | 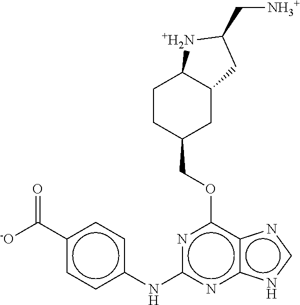 | −12.559 | −11.613 |
| 73 | 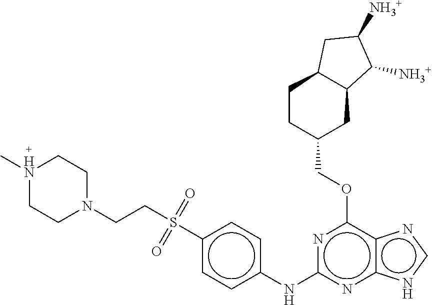 | −12.556 | −12.348 |
| 74 | 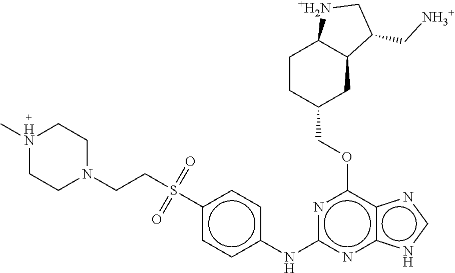 | −12.521 | −13.077 |
| 75 | 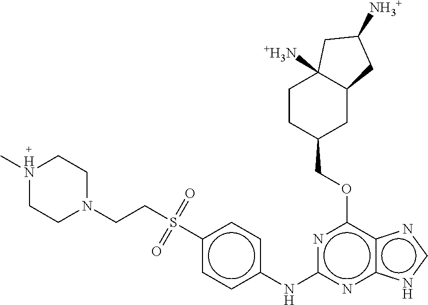 | −12.516 | −12.043 |
| 76 | 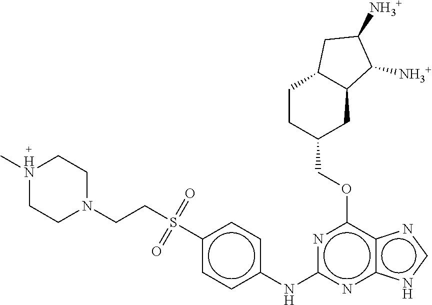 | −12.495 | −12.209 |
| 77 | 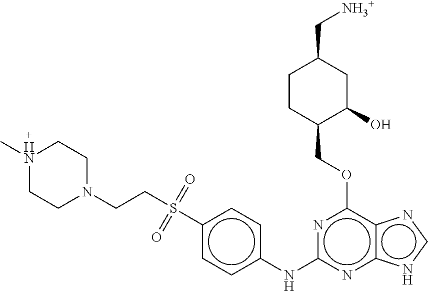 | −12.488 | −13.205 |
| 78 | 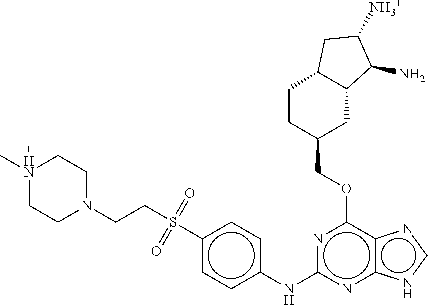 | −12.479 | −13.261 |
| 79 | 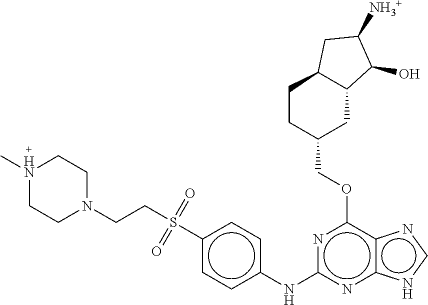 | −12.477 | −13.605 |
| 80 | 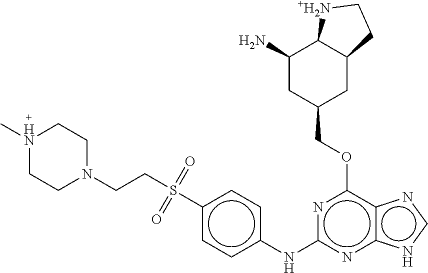 | −12.467 | −12.766 |
| 81 | 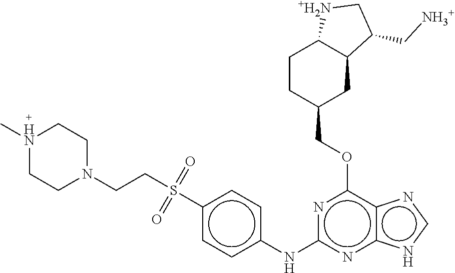 | −12.456 | −12.518 |
| 82 | 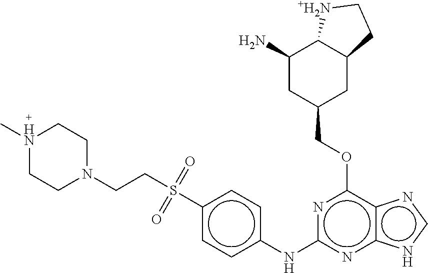 | −12.455 | −12.988 |
| 83 | 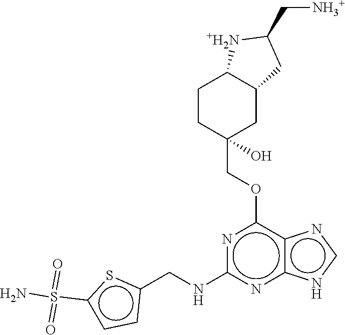 | −12.453 | −12.597 |
| 84 | 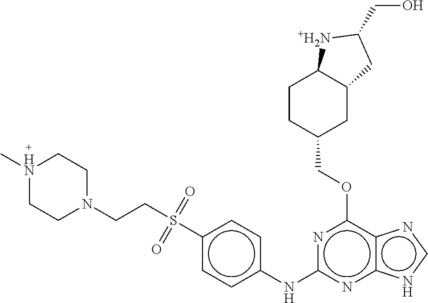 | −12.443 | −12.152 |
| 85 | 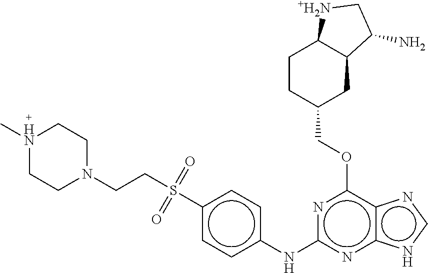 | −12.44 | −12.893 |
| 86 | 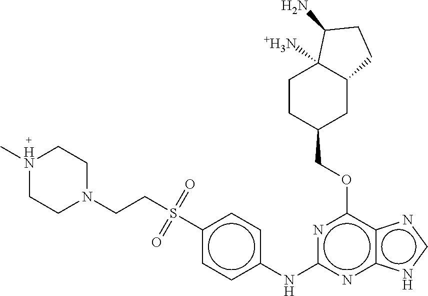 | −12.44 | −11.928 |
| 87 | 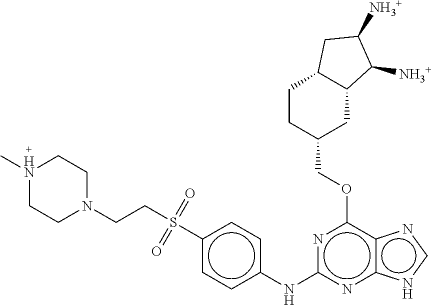 | −12.398 | −12.348 |
| 88 | 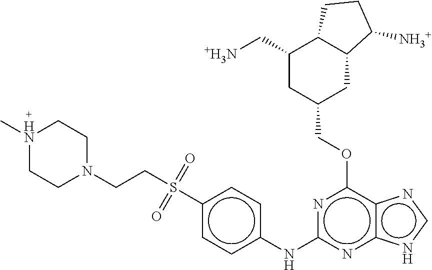 | −12.398 | −12.979 |
| 89 | 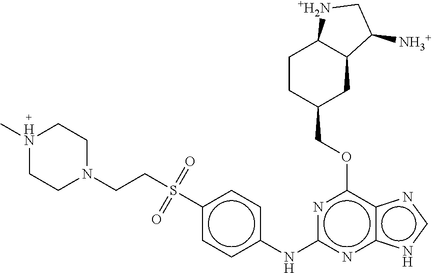 | −12.39 | −12.699 |
| 90 | 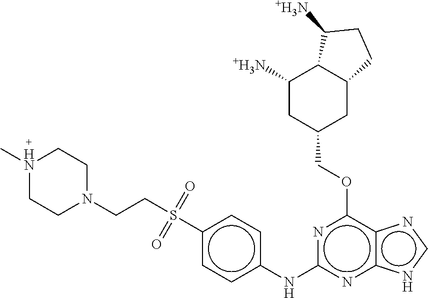 | −12.385 | −12.844 |
| 91 | 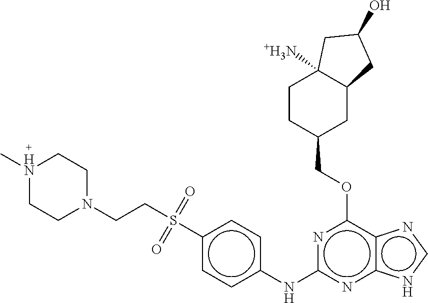 | −12.383 | −12.818 |
| 92 | 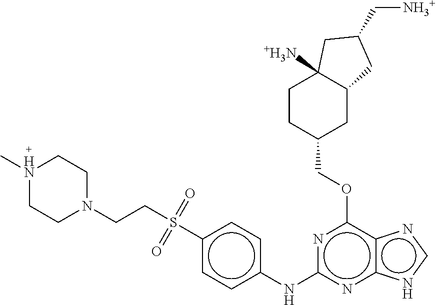 | −12.378 | −12.614 |
| 93 | 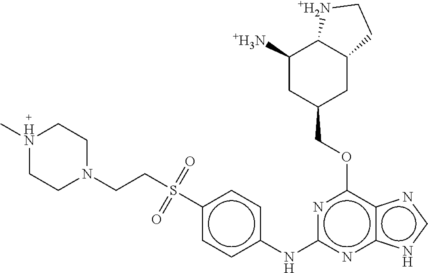 | −12.368 | −12.928 |
| 94 | 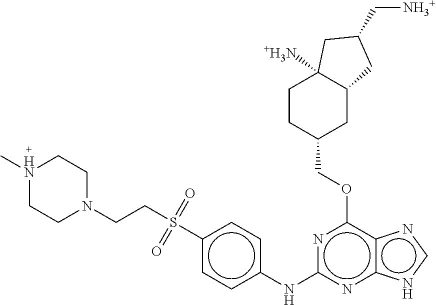 | −12.368 | −12.656 |
| 95 | 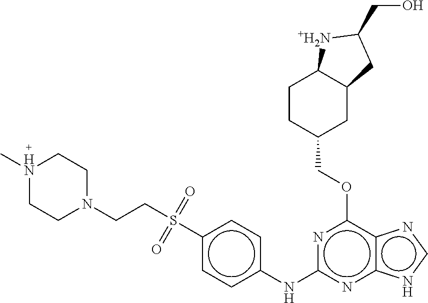 | −12.367 | −12.592 |
| 96 | 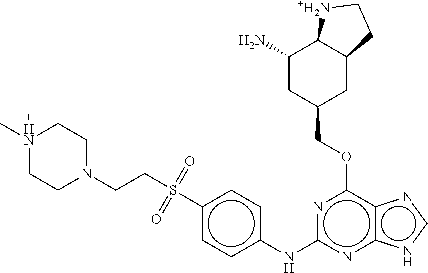 | −12.36 | −12.602 |
| 97 | 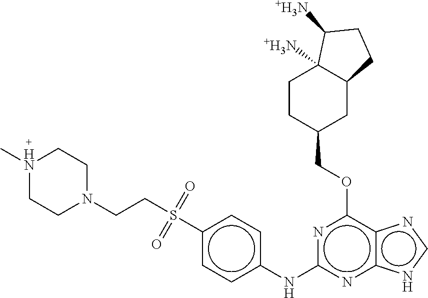 | −12.36 | −12.505 |
| 98 | 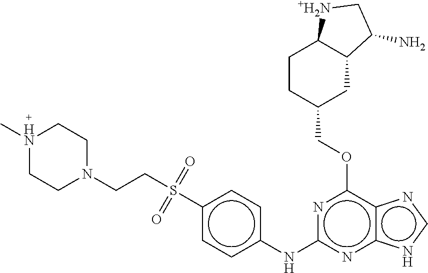 | −12.356 | −13.53 |
| 99 | 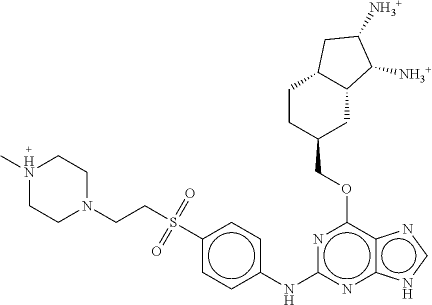 | −12.355 | −13.024 |
| 100 | 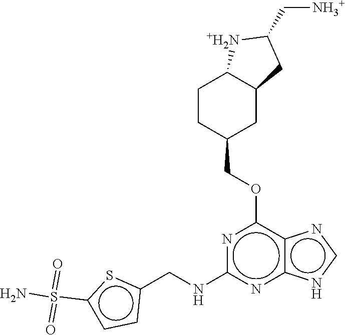 | −12.348 | −12.714 |

While the invention has been described with reference to preferred embodiments, the invention is not or intended by the applicant to be so limited. A person skilled in the art would readily recognize and incorporate various modifications, additional elements and/or different combinations of the described components consistent with the scope of the invention as described herein.

We claim:

1. A compound having structural formula I, II, III or IV or a pharmaceutically acceptable salt, hydrate, solvate, polymorph or prodrug thereof: 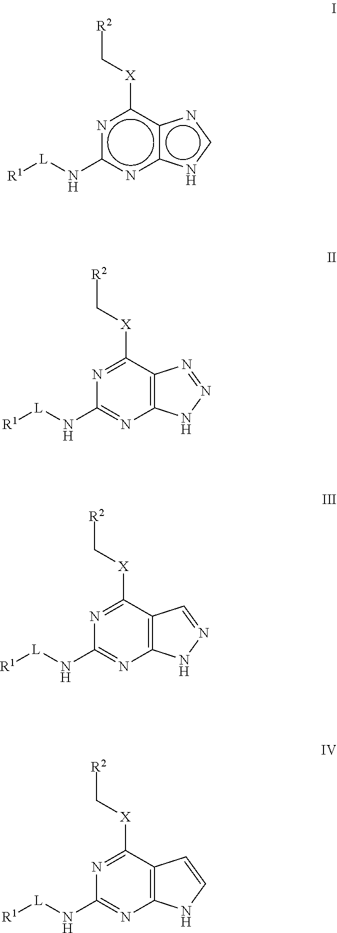
wherein X is O or N; L is absent or C₁-C₄ alkylene; R¹ is an optionally substituted aryl or heteroaryl; and R² is an optionally substituted cycloalkyl, bicycloalkyl, tricycloalkyl, cycloheteroalkyl, bicycloheteroalkyl or tricycloheteroalkyl.

2. The compound of claim 1, wherein R¹ is optionally substituted phenyl or thiophenyl.

3. The compound of claim 2, wherein L is absent and R¹ is phenyl substituted with one or more of —SO₂NH₂, —SOCH₃ or —CO₂H, or R¹ is 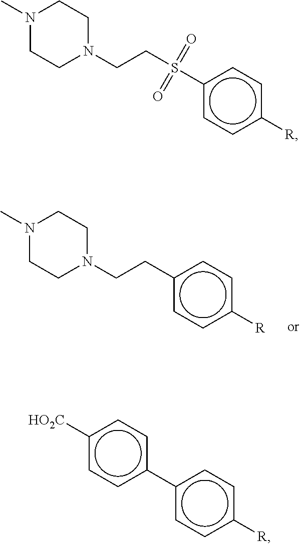
wherein R is remainder of the compound.

4. The compound of claim 1, wherein R¹ is 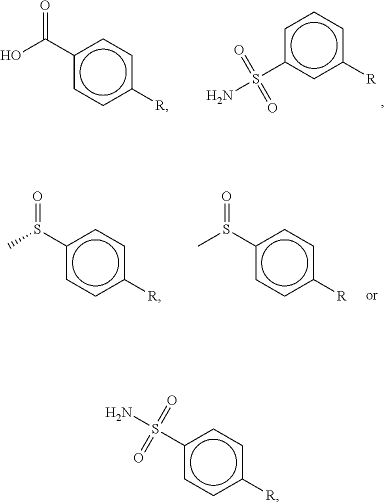
wherein R is remainder of the compound.

5. The compound of claim 2, wherein R¹ is thiophenyl substituted with —SO₂NH₂ or L and R¹ are 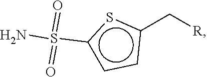
wherein R is remainder of the compound.

6. The compound of claim 1, wherein R² is an optionally substituted C₅-C₈ cycloalkyl, C₈-C₁₄ bicycloalkyl or 8- to 14-membered bicycloheteroalkyl containing one to four heteroatoms each selected from the group consisting of an oxygen atom, a nitrogen atom and a sulfur atom.

7. The compound of claim 1, wherein R² is optionally substituted cyclohexyl, bicyclo[4.3.0]nonyl, bicyclo[3.3.0]octyl, octahydro-1H-indolyl or octahydro-1H-cyclopenta[b]pyridinyl.

8. The compound of claim 7, wherein R² is optionally substituted 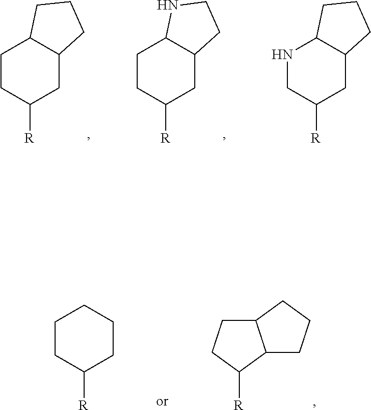
wherein R is remainder of the compound.

9. The compound of claim 1, wherein R² is optionally substituted with one or more of —NH₂, —CH₂NH₂, —OH, —CH₂OH, —CO₂H, 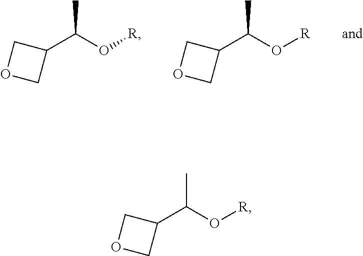
wherein R is remainder of the compound.

10. The compound of claim 1, wherein R² is 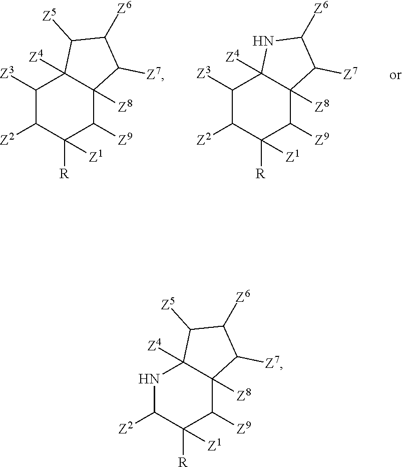
wherein
Z¹ is —H or —OH;
Z² is —H or 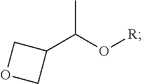
Z³ is —H, —NH₂ or —CH₂NH₂;
Z⁴ is —H or —NH₂;
Z⁵ is —H, —NH₂ or —CO₂H;
Z⁶ is —H, —NH₂, —OH, —CH₂NH₂ or —CH₂OH;
Z⁷ is —H, —NH₂, —OH, —CO₂H, —CH₂NH₂ or —CH₂OH;
Z⁸ is —H;
Z⁹ is —H, —NH₂ or —OH; and
R is remainder of the compound.

11. The compound of claim 10, wherein R² is 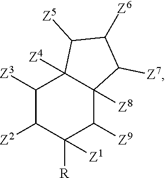
wherein Z¹ and Z² are each —H; Z³ is —H, —NH₂ or —CH₂NH₂; Z⁴ is —H or —NH₂; Z⁵ is —H, —NH₂ or —CO₂H; Z⁶ is —H, —NH₂, —OH, —CH₂NH₂ or —CH₂OH; Z⁷ is —H, —NH₂ or —OH; Z⁸ is —H; Z⁹ is —H or —NH₂; and R is remainder of the compound.

12. The compound of claim 10, wherein R² is 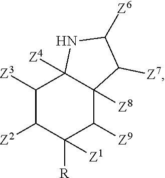
wherein Z¹ is —H or —OH; Z² is —H or 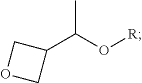
Z³ is —H or —NH₂; Z⁴ is —H; Z⁶ is —H, —CH₂NH₂ or —CH₂OH; Z⁷ is —H, —OH, —NH₂, —CO₂H or —CH₂NH₂; Z⁸ is —H; Z⁹ is —H or —OH; and R is remainder of the compound.

13. The compound of claim 10, wherein R² is 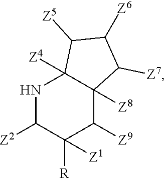
wherein Z¹, Z², Z⁴ to Z⁶, Z⁸ and Z⁹ are each —H; Z⁷ is —H or NH₂; and R is remainder of the compound.

14. The compound of claim 6, wherein R² is 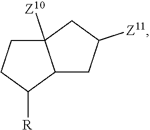
wherein Z¹⁰ is —H or —OH; Z¹¹ is —H or —CH₂NH₂; and R is remainder of the compound.

15. The compound of claim 6, wherein R² is 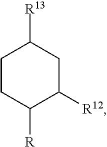
wherein Z¹² is —H or —OH;
R¹³ is —H or —CH₂NH₂; and R is remainder of the compound.

16. The compound of claim 1, wherein R² is

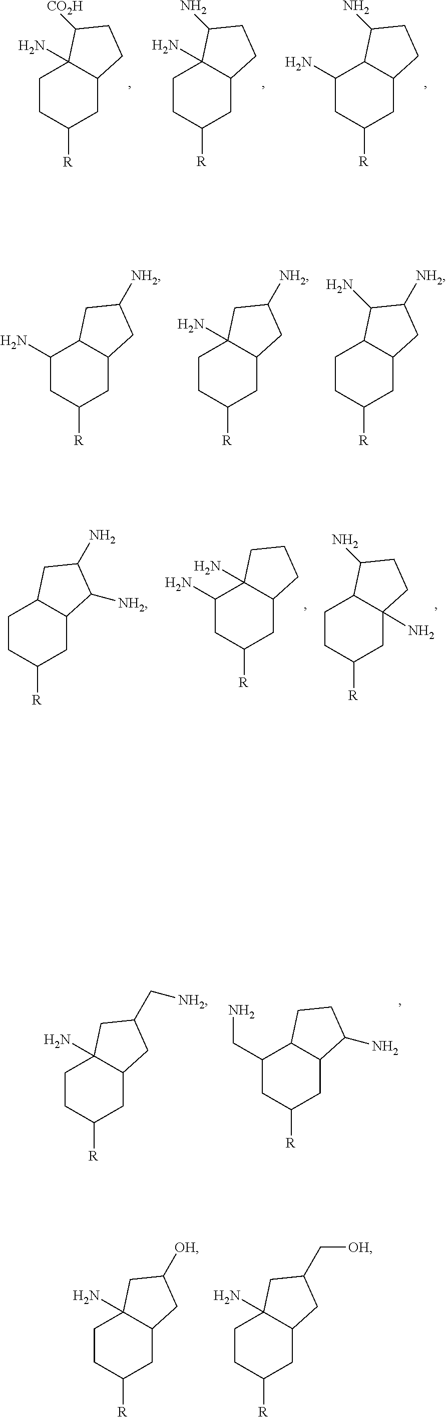

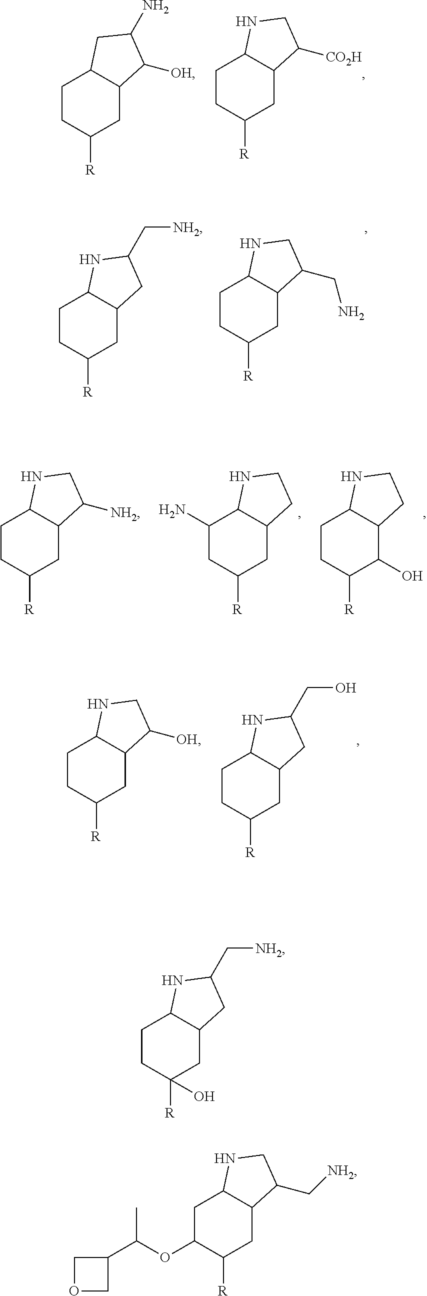

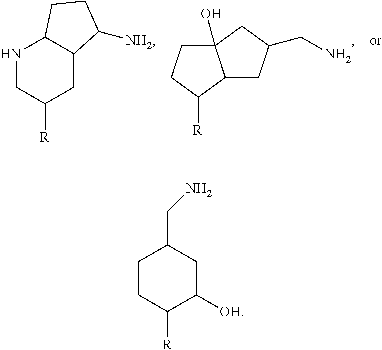

17. The compound of claim 1, wherein the compound is one of the compounds listed in Tables 1 and 2, excluding flavopiridol, dinaciclib, purvalanol A and NU6102.

18. The compound of claim 1, wherein the compound is coupled to a fluorescent, biotin or proteolysis-targeting chimera (PROTAC) moiety, optionally wherein the fluorescent, biotin or PROTAC moiety is coupled to RI.

19. A method for treating cancer, the method comprising administering to a patient the compound as defined by claim 1.

20. The method of claim 19, wherein the compound is for binding to an active site of a complex comprising a cyclin-dependent kinase and Spy1, wherein the cyclin-dependent kinase is Cdk1 or Cdk2.

21. The method of claim 19, wherein the cancer is a breast, brain, liver, blood, prostate, endometrial and ovarian cancer.

22. A method for identifying, imaging or quantifying a Spy1-Cdk2 complex in a sample, the method comprising contacting the sample with the compound as defined by claim 18, wherein the compound is coupled to the fluorescent moiety at R¹.

23. A method for identifying a protein target of the compound as defined by claim 18, the method comprising contacting the compound with a sample, wherein the compound is coupled to the biotin moiety at R¹.

24. A pharmaceutical composition for treating cancer, the composition comprising the compound as defined by claim 1 and a pharmaceutically acceptable excipient.

25. The pharmaceutical composition of claim 24, wherein the cancer is a breast, brain, liver, blood, prostate, endometrial and ovarian cancer.
